(19) United States
(12) Patent Application Publication
(10) Pub. No.: US 2008/0262321 A1
(43) Pub. Date: Oct. 23, 2008

(54) Early Detection of Harmful Agents: Method, System and Kit

(75) Inventors: Mendy Erad, Los Altos, CA (US); Yosi Shacham-Diamand, Zikhron-Yaakov (IL); Shimshon Belkin, Kiryat Ono (IL); Arthur Rabner, Natania (IL); Yariv Erad, Kidron (IL); Rami Pedahzur, Doar-Na Harei Yehuda (IL); Yehuda Yarmut, Moshava Yokneam (IL)

Correspondence Address: Martin D. Moynihan;PRTSI, Inc., P.O. Box 16446, Arlington, VA, 22215, US

(73) Assignees: Ramot At Tel Aviv University Ltd., Tel-Aviv (IL); Yissum Research Development Company of the Hebrew University of Jerusalem, Jerusalem (IL)

(21) Appl. No.: 11/659,635

(22) PCT Filed: Aug. 4, 2005
(86) PCT No.: PCT/IL05/00843

§ 371 Date: Mar. 27, 2008

Related U.S. Application Data
(60) Provisional application No. 60/599,033, filed on Aug. 6, 2004.
(60) Provisional application No. 60/625,057, filed on Nov. 5, 2004.

(51) Int. Cl. G01N 33/00; C12Q 1/00; A61M 5/20; B01L 3/00; G08B 21/12; A61B 5/00; G01N 21/76; C12M 1/34

(52) U.S. Cl. 600/301; 604/191; 422/61; 422/52; 340/573.1

(57) ABSTRACT

A portable system for detecting agents present in a sample is disclosed. The system comprises a sensing device, having a substrate formed with a plurality of reaction chambers and a plurality of channels interconnecting at least a portion of the plurality of reaction chambers, wherein at least a portion of the plurality of reaction chambers comprises a sensor, capable of generating a detectable signal when exposed to the agents. The system further comprises a detector, which receives signals from the sensing device and provides an image of sensors generating the optical signals. The portable system is connected to a communication network via a communication unit.

### FIELD AND BACKGROUND OF THE INVENTION

The present invention relates to harmful agents detection and, more particularly, to early detection and warning of presence or diffusion of harmful agents, such as, but not limited to, chemical, biological or radioactive (CBR) agents.

Spread of harmful agents presents a major concern to the future of mankind. This is due to the non-local nature of the spread whereby the source of such a threat may be relatively localized, but its effect can appear in many other locations.

Typically, a large time delay occurs between the occurrence of a harmful agent incident, and the time at which the appropriate authorities are able to conclude that a threat is underway. For example, when a region, populated by a particular community, becomes contaminated, e.g., by a cargo spill or by a deliberate act of terrorism, a certain period of time lapses before the contamination or the effect thereof is noticed.

The time delay is either due to the nature of the harmful agent (e.g., gestation period of a biological agent, or artificial delay), or because of a failure in concluding that an observed pattern of events is indicative of the harmful agent incident. It is recognized that if the time delay to detection is long, the incident is likely to be aggravated because the contamination may diffuse to a neighboring population.

Harmful agents may spread to large areas in many ways and many combinations of different ways, such as transport in a medium (e.g., air, surface water, groundwater, soil), transport in via a vehicle (missiles, population movement) reproduction, multiplication, bioaccumulation and the like. This diversity of options makes the propagation patterns of harmful agents almost unpredictable. Unlike conventional bombs, for which the range of damage is limited even if an explosion occurs in a heavily populated location, the damage caused by harmful agent attack can spread, grow with time and cover huge, and in extreme cases, almost unlimited areas.

An utmost pressing problem involving spread of harmful agents is that of early detection and warning. In an era where harmful agent attacks at one or more locations either globally or within a country are possible, it is desirable to have a surveillance system capable of detecting and locating the attack at an early stage. The first priority in the management of harmful agent release events involves detecting that harmful agents have been released, and warning the appropriate authorities of the event. Once the identity and physical properties of the harmful agent that has been released are ascertained, effective measures, such as definition of outer perimeter, initiation of decontamination procedures, or formulation of neutralization plans, can be taken. Whether the release occurs as a result of enemy activity or as a result of an accident in a domestic facility, a prompt detection is crucial to minimize injury and loss of life.

Known in the art are monitoring devices which are designed to be placed in a particular vulnerable medium, such as a water reservoir or an air condition system. Such monitoring devices can only alert when a harmful agent incident occurs in the medium in which they are installed. Threat scenarios, however, are unpredictable in nature and incidents may occur simultaneously in more than one place and/or more than one medium. In particular, such monitoring devices are practically useless for alerting of a silent or even explosive release of a harmful agent in an open environment.

U.S. Pat. No. 6,293,861 discloses a building protection system responsive to the release of airborne agents both outside and inside of a building. An array of sensors surrounding the perimeter of the building is triggered to provide an indication when an external release has occurred. Upon initial detection of a release, a central processor connected to receive a release signal from the sensor shuts down all external air exchanges for the building and activates an over-pressure system for the building interior to insure that contaminated external air does not enter. Exterior sampling inlets are monitored to determine if high agent concentrations exist as a confirmation of the indication given by the perimeter sensors. When an internal attack occurs in the entrance area of the building, an internal sensor transmits an appropriate signal to the central processor, which closes off the entrance area, exhausts air from the closed area through a filter and activates the over-pressure system. Sampling inlets connected in various areas within the building monitor these areas to determine whether they have been contaminated and the concentration and type of contaminating agent. The processor can activate a decontaminant spray system to decontaminate the contaminated areas.

The above system can be typically employed in particular buildings, such as key military sites, which are equipped or designed well in advance to deal with the use of harmful agents. The built-in fixed sensors, which are generally limited to sensing one area of the building, may be too expensive to be placed in all desired areas of the building. Other facilities, such as hotels, department stores, shopping malls and the like, are more susceptible to harmful agents, lacking even the aforementioned fixed sensors.

U.S. Pat. No. 6,701,772 discloses a system for detecting harmful agents in buildings. The system includes a moving detector which traverses spaces in the buildings, detects presence of harmful agents and transmits data to a receiver. The data includes both the type of harmful agent and the location at which the agent was detected.

U.S. Pat. No. 6,710,711 discloses a method of identifying hazards occurring within an area by combining syndromic data with modeling and simulation operation. The actual syndromic data is compared with the simulation results to determine whether or not the syndromic data correlates with the simulation results.

The above prior art attempts have been exclusively directed to provide a local scale detection of harmful agents, typically in a predetermined cite, by employing either a fixed arrangement of sensors, or a self propelled detector to locally cover a confined area. However, because the technological and financial problems associated with adaptation of known techniques a global scale, the harmful agent detection solutions provided by prior art are far from being satisfactory.

With respect to detection means, much industrial and academic effort is presently directed at the development of detection micro systems combining electrical, mechanical and/or optical/electrooptical components, commonly known as Micro Electro Mechanical Systems (MEMS). MEMS are fabricated using integrated circuit batch processing techniques and can range in size from micrometers to millimeters. These systems can sense, control and actuate on the micro scale, and function individually or in arrays to generate effects on the macro scale.

The development of miniaturized devices for chemical analysis and for synthesis and fluid manipulation is motivated by the prospects of improved efficiency, reduced cost and enhanced accuracy. Efficient, reliable manufacturing processes are a critical requirement for the cost-effective, high-volume production of devices that are targeted at high-volume, high-throughput test markets.

In the most general form, MEMS consist of mechanical microstructures, microsensors, microactuators and electronics which are integrated into a single device or platform (e.g., on a silicon chip). The microfabrication technology enables fabrication of large arrays of devices, which individually perform simple tasks but in combination can accomplish complicated functions.

One type of MEMS is a microfluidic device. Microfluidic devices include components such as channels, reservoirs, mixers, pumps, valves, chambers, cavities, reaction chambers, heaters, fluidic interconnects, diffusers, nozzles, and other microfluidic components. These microfluidic components typically have dimensions which range between several micrometers to several millimeters. The small dimensions of such components minimize the physical size, the power consumption, the response time and the waste of a microfluidic device as compared to other technologies.

In the area of life science, microfluidic devices are used in DNA chips, protein chips and total analysis systems (also known as lab-on-chip). The use of a microfluidic device in the fabrication process of a microchip facilitates the production of small and high-density spots on the substrate. Since only a small amount of solution is needed to make one chip, the cost of chip production is substantially reduced. In addition, a microfluidic device can created spots in consistent quantities and with uniform configurations, so as to enable highly accurate comparisons between spots.

Microfluidic devices are typically used in genetic, chemical, biochemical, pharmaceutical, biomedical, chromatography, medical, radiological and environmental applications. For example, in environmental applications, such devices are used for detecting hazardous materials or conditions, air or water pollutants, chemical agents, biological organisms or radiological conditions. The genetic and biochemical applications include testing and/or analysis of molecules, or reactions between such molecules in microfluidic devices.

In a microfluidic device, a plurality of determinations may be performed concurrently and/or consecutively. By having channels that have ultra small cross-sections, operations can be carried out with very small volumes. In addition, by having very sensitive detection systems, very low concentration of a detectable label can be employed. This allows for the use of very small samples and small amounts of reagents.

Droplet microfluidics refers to the set of technologies that are being developed for manipulating very small, substantially uniform, liquid drops, micro- to nano-liters in volume, which are supported on a solid surface, sandwiched between two solid plates or sucked into a solid channel. The manipulations include moving the droplets around, making them coalesce, and breaking them up. These technologies have a promising potential for developing commercially viable droplet-based microfluidic platforms for biotechnology and other applications. One of the reasons is that the smaller the length scale over which transport processes (convection, diffusion and reaction) take place, the faster the completion time of the process. As such, the drive toward high-throughput screening and diagnostics requires the concomitant development of associated microfluidic enabling technologies.

Droplet microfluidics may be employed in the area of biochemical and biophysical investigations of single cells. Knowledge of cell activity may also be gained by measuring and recording electrical potential changes occurring within a cell, which changes depend on the type of cells, age of the culture and external conditions such as temperature or chemical environment. Thus, precisely controlling the physical and chemical environment of a cell under study significantly enhances the value of the research. In addition, as further detailed hereinunder, cells activity can also exploited for the purpose of detecting and/or identifying chemicals in a sample.

The ability to sense, analyze, monitor and/or control transport of fluid through or from a microchannel is one of the fundamental properties required for all the above applications.

An objective in developing new techniques is not only to be able to selectively identify target compounds but to be able to assay large numbers of samples. Yet, there remain problems in detecting and measuring low levels of compounds conveniently, safely and quickly.

One of the favored approaches of analyzing, detecting and/or monitoring substances involves the use of light. Traditional methods involve illuminating a sample with light and using absorbance or scattering characteristics of the sample to analyze the agent present therein. Typically, light based methods utilize one or more luminescent materials, such as fluorescent materials, either as labels or as reporters. Additionally, the agent of interest can also have luminescent properties.

When a fluorescent atom or molecule absorbs light, electrons are boosted to a higher energy shell of an unstable excited state. During the lifetime of excited state (typically 1-10 nanoseconds) the fluorescent atom undergoes conformational changes and is also subject to a multitude of possible interactions with its molecular environment. The energy of excited state is partially dissipated, yielding a relaxed singlet excited state from which the excited electrons fall back to their stable ground state, emitting light of a specific wavelength. The emission spectrum is shifted towards a longer wavelength than its absorption spectrum. The difference in wavelength between the apex of the absorption and emission spectra of the fluorescent atom (also referred to as the Stokes shift), is typically small.

In a fluorescent material, not all the molecules initially excited by absorption return to the ground state by fluorescence emission. Other processes such as collisional quenching, fluorescence resonance energy transfer and intersystem crossing may also depopulate the excited state. A ratio of the number of fluorescence photons emitted to the number of photons absorbed, called “fluorescence quantum yield,” is a measure of the relative extent to which these processes occur. For fluorescent materials which are commercially available, only a small portion (about 0.1%) of the absorbed light is actually emitted.

The low fluorescence quantum yield and the small separation between the absorption and emission spectra, require the usage of spectral discrimination methods to allow a clear detection. Typically, the discrimination methods utilize a set of filters on the excitation path and emission path of a fluorescence detection system. Such filters were greatly developed during the past years, and are being manufactured by various companies.

Heretofore, attempt to developed microfluidic devices for the purpose of detecting chemical or biological agents, resulted in only partial success.

U.S. Pat. No. 6,614,030 discloses an optical detection device for identifying and detecting fluorophores in during operations involving fluorescent signals. The optical detection device includes an excitation light source, and an optical setup for guiding excitation light and emission light. In use, the housing of the device is accurately moved over a small area in relation to a channel in a microfluidic device.

U.S. Pat. No. 6,602,702 discloses a system for the rapid characterization of multi-agent fluids. The system employs a sensor array, formed of a plurality of cavities, each trapping a chemically sensitive particle, which is configured to produce light when a receptor coupled to the particle interacts with an agent of the fluid. The agents within the fluid are then characterized by pattern recognition techniques.

U.S. Pat. No. 6,551,838 discloses a device having a plurality of reservoirs covered by barrier layers which can be disintegrated or permeabilized to expose the isolated contents to the one or more environmental components. The reaction between the contents of the reservoirs and the environmental components generate a signal which is detected by conventional technique.

Several attempts have been made to incorporate biological materials as biosensors capable of sensing physical or chemical environmental conditions in microfluidic devices.

Generally, a biosensor is a device that qualifies and/or quantifies a physiological or biochemical signal. Biosensors have been developed to overcome some of the shortcomings of the classical agent detection techniques. Good biosensing systems are characterized by specificity, sensitivity, reliability, portability, ability to function even in optically opaque solutions, real-time analysis and simplicity of operation. Biosensors couple a biological component with an electronic transducer and thus enable conversion of a biochemical signal into a quantifiable electrical response.

The function of the biosensor depends on the biochemical specificity of the biologically active material. Enzymes, antibodies, aptamers, DNA, receptors, organelles and microorganisms as well as plant cells or tissues have been used as biological sensing elements. The most commonly used biological element in the construction of biosensors are enzymes, due to their high specific activities as well as high analytical specificity. Purified enzymes are, however, expensive and unstable, thus limiting their applications in the field of biosensors.

The use of whole cells as the biosensing element negates the lengthy procedure of enzyme purifications, preserves the enzymes in their natural environment and protects it from inactivation by external toxicants such as heavy metals. Whole cells also provide a multipurpose catalyst especially when the process requires the participation of a number of enzymes in sequence. Whole cells have been used either in viable or non-viable form. Viable microbes, for example, can metabolize various organic compounds resulting in various end products like ammonia, carbon dioxide, acids and the like, which can be monitored using a variety of transducers [Burlage (1994) Annu. Rev. Microbiol. 48: 291-309; Riedel (1998) Anal. Lett. 31:1-12; Arikawa (1998) Mulchandani, Rogers (Eds.) Enzyme and Microbial Biosensors: Techniques and Protocols. Humanae Press, Totowa, N.J., pp. 225-235; and Simonian (1998) Mulchandani, Rogers (Eds.) Enzyme and Microbial Biosensors: Techniques and Protocols. Humanae Press, Totowa, N.J. pp: 237-248].

The selection of microbial culture which corresponds well with a spectrum of compounds present in the sample is of significant importance.

A number of selection approaches are known in the art. For example, adaptation of a microbe for induction of desirable metabolic pathways and uptake systems can be effected by cultivation in a medium containing appropriate substrates [Di Paolantonio and Rechnitz (1982) supra; Riedel (1990) Anal. Lett. 23:757-770; Fleschin (1998) Prep. Biochem. Biotechnol. 28:261-269]. Specifically, for the biochemical degradation of complex substrates such as mixtures of phenols, the use of activated sludge obtained from waste treatment plants can serve as an acclimatized mixed microbial consortium as compared to pure cultures [Joshi and D'souza (1999) J. Environ. Sci. Health Part A Eviron. Sci. Engng. 34:1689-1700].

Alternatively, when a single cell does not contain all enzymes necessary for a sequential set of reactions a mixture of microbial cultures can be used. Thus, Gluconobacter oxydans containing glucose oxidase has been used in conjunction with saccharomyces cerevisiae cells containing periplasmic invertase or permeabilized Kluyveromyces marxianus cells containing intracellular β-galactosidase, in the fabrication of a sucrose and a lactose biosensor, respectively [Svitel (1998) Biotechnol. Appl. Biochem. 27:153-158]. Note, the major drawback of such an approach is the need to maintain at least two cultures of microorganisms on a single sensor which may prove problematic such as due to different nutritional needs.

Microbial biosensors based on light emission from luminescent bacteria are also utilized in agent detection. Bioluminescent bacteria are found in nature, their habitat ranging from marine to terrestrial environments. Bioluminescent whole cell biosensors have also been developed using genetically engineered microorganisms for the monitoring of organic, pesticide and heavy metal contamination. The microorganisms used in these biosensors are typically produced with an exogenous plasmid into which a reporter gene under the control of an inducible promoter of interest is placed.

Following are prior art technologies incorporating biosensors in microfluidic devices.

U.S. Pat. No. 6,436,698 is directed at automatic measurement of water toxicity, using luminescent microorganisms living in freshwater. Test samples are injected using a needle into multi-well plate containing the luminescent microorganisms and, after a lapse of certain times from the injection, luminosity is detected by a sensor.

U.S. Pat. No. 6,117,643 is directed at detection of pollutants, explosives and heavy-metals. A bioreporter, capable of metabolizing a particular substance to emit light, is placed in a selectively permeable container. When the light is emitted, an optical application specific integrated circuit generates an electrical signal which indicates the concentration of the substance.

U.S. Pat. No. 6,133,046 teaches the use of a fixed electrode and a moving electrode, whereby the surfaces of the electrodes bound a ligand of the agent to be detected (e.g., an antibody, whereby the agent is an antigen or a hapten, a receptor whereby the agent is a receptor, etc.). When a sample is placed between the electrodes, an electric signal is generated, depending on whether or not the agent is present.

Additional prior art of relevance include: U.S. Pat. Nos. 6,638,752, 6,638,483, 6,636,752, 6,632,619, 6,627,433, 6,630,353, 6,620,625, 6,544,729, 6,537,498, 6,521,188, 6,453,928, 6,448,064, 6,340,572 and 5,922,537.

The above technologies suffer from many limitations. For example, in most prior art systems, the optical setup which is large, bulky and generally unsuitable for field use. In addition, there is the problem of obtaining a reliable optical signal, in effect compromising maximizing the signal from the detectable material while minimizing the background signal. Furthermore, in prior art systems which are based on mechanical scan (e.g., moving electrode, moving light ray or moving sample), inaccurate readings may occur due to misalignment of the various components. With respect to the sensing process, it is difficult to generate transport of the sample in the channels and to distinguish between signals arriving from different locations.

There is thus a widely recognized need for, and it would be highly advantageous to have a method, system and kit for detecting and/or identifying chemical, biological or radioactive agents, devoid of the above limitations. The present invention provides solutions to the problems associated with prior art techniques aimed at early detection and warning of presence or diffusion of harmful agents.

### SUMMARY OF THE INVENTION

According to one aspect of the present invention there is provided a portable system for detecting agents present in a sample, the system comprising: a sensing device having a substrate formed with a plurality of reaction chambers and a plurality of channels interconnecting at least a portion of the plurality of reaction chambers, wherein at least a portion of the plurality of reaction chambers comprises a sensor, capable of generating a detectable signal when exposed to the agents; a detector capable of receiving signals from the sensing device and providing an image of sensors generating the detectable signals; and a communication unit, for connecting the portable system to a communication network.

According to further features in preferred embodiments of the invention described below, the system further comprises a positioning unit, for determining a location of the system, wherein the communication unit is designed to transmit the location over the communication network.

According to still further features in the described preferred embodiments the system further comprises a personal accessory device, wherein the sensing device, the detector and the communication unit are integrated with or installed in a body of the personal accessory device.

According to still further features in the described preferred embodiments the system further comprises a backup power source.

According to still further features in the described preferred embodiments the system is powered by a power source of the personal accessory device.

According to still further features in the described preferred embodiments the system further comprises an activation unit for activating or selecting an operational mode of the system.

According to still further features in the described preferred embodiments the system further comprises a user interface, wherein the activation unit is controllable by the user interface.

According to still further features in the described preferred embodiments the system further comprises a portable power source.

According to still further features in the described preferred embodiments the system further comprises a user display.

According to still further features in the described preferred embodiments the system further comprises a supplementary sensing unit capable of continuously monitoring environmental conditions, and generating a signal to the activation unit to activate the system when the environmental conditions meet a predetermined set of criteria.

According to still further features in the described preferred embodiments the system is identifiable by an identification code, and further wherein the communication unit is operable to transmit the identification code over the communication network.

According to still further features in the described preferred embodiments the system further comprises detection hardware.

According to still further features in the described preferred embodiments the system further comprises vital signs measuring unit for measuring vital signs of a mammal carrying the system.

According to still further features in the described preferred embodiments the system further comprises an atmospheric condition measuring unit for measuring at least one atmospheric condition.

According to still further features in the described preferred embodiments the system further comprises an armored encapsulation.

According to still further features in the described preferred embodiments the system further comprises an image capturing unit.

According to still further features in the described preferred embodiments the system further comprises an input-output audio unit.

According to still further features in the described preferred embodiments the system further comprises a nucleic acid amplification unit.

According to still further features in the described preferred embodiments the system further comprises a shape detector.

According to still further features in the described preferred embodiments the system further comprises a motion detector.

According to still further features in the described preferred embodiments the system further comprises a data processor, supplemented by an algorithm for receiving image information from the detector and determining presence or level of the agents.

According to still further features in the described preferred embodiments the system further comprises a control unit for sending control signals to the sensing device.

According to still further features in the described preferred embodiments the system further comprises a temperature control unit for controlling a temperature of the sensing device and/or the detector.

According to still further features in the described preferred embodiments the system further comprises a light source for emitting excitation light so as to excite the sensor to thereby emit the optical signal.

According to still further features in the described preferred embodiments the detector is selected from the group consisting of a sound acoustic wave resonator, a surface plasmon resonance detector and a planar light detector.

According to still further features in the described preferred embodiments the system further comprises a temperature control unit for controlling a temperature of the sensing device, the planar light detector and/or the light source.

According to still further features in the described preferred embodiments the system further comprises at least one selective filter positioned between the sensing device and the planar light detector, the at least one selective filter being capable of transmitting the optical signals and preventing transmission of the excitation light.

According to still further features in the described preferred embodiments the system further comprises an optical focusing device for focusing the optical signal on the planar light detector.

According to still further features in the described preferred embodiments the system further comprises a transport mechanism for actuating transport of a sample fluid in the plurality of fluid channels, thereby to fill the plurality of reaction chambers with the sample fluid.

According to still further features in the described preferred embodiments the system further comprises a draining system and further wherein the transport mechanism is capable of maintaining a continues flow of the sample fluid in the plurality of fluid channels thereby to continuously replace the sample fluid in the plurality of reaction chambers.

According to still further features in the described preferred embodiments the system further comprises an electronic circuitry designed and constructed for controlling the plurality of micro-pumps.

According to still further features in the described preferred embodiments the system further comprises electronic circuitry for controlling flow rate of the sample fluid.

According to still further features in the described preferred embodiments the sample is a gas sample.

According to still further features in the described preferred embodiments the system further comprises a mechanism for binding components of the gas sample to a liquid phase.

According to still further features in the described preferred embodiments the sample is a solid sample.

According to still further features in the described preferred embodiments the system further comprises a mechanism for binding components of the solid sample to a liquid phase.

According to another aspect of the present invention there is provided a kit for detecting agents present in a sample, the kit comprising: (a) a portable detection system, comprising: a sensing device having a substrate formed with a plurality of reaction chambers and a plurality of channels interconnecting at least a portion of the plurality of reaction chambers, wherein at least a portion of the plurality of reaction chambers comprises a sensor, capable of generating a detectable signal when exposed to the agents; a detector capable of receiving signals from the sensing device and providing an image of sensors generating the optical signals; and a communication unit, for connecting the portable system to a communication network; and (b) a sampling device being connectable to the detection system and capable of sampling fluids from the environment and delivering the fluids to the sensing device.

According to further features in preferred embodiments of the invention described below, the portable detection system further comprises a positioning unit, for determining a location of the portable detection system, wherein the communication unit is designed to transmit the location over the communication network.

According to still further features in the described preferred embodiments the portable detection system further comprises an activation unit for activating or selecting an operational mode of the portable detection system.

According to still further features in the described preferred embodiments the user interface is designed and configured to generate at least one sensible signal being indicative of an operative status and/or impermeability level of the portable detection system.

According to still further features in the described preferred embodiments the portable detection system further comprises a portable power source.

According to still further features in the described preferred embodiments the user interface is designed and configured to generate at least one sensible signal being indicative of a power level of the power source.

According to still further features in the described preferred embodiments the user interface is powered by an additional power source, hence being operative when the power source is not operative.

According to still further features in the described preferred embodiments the activation unit is in communication with a central location and being controllable thereby, hence allowing a remote activation or operational mode selection of the portable detection system.

According to still further features in the described preferred embodiments the portable detection system further comprises a supplementary sensing unit capable of continuously monitoring environmental conditions, and generating a signal to the activation unit to activate the portable detection system when the environmental conditions meet a predetermined set of criteria.

According to still further features in the described preferred embodiments the kit further comprises vital signs measuring unit for measuring vital signs of a mammal carrying the kit.

According to still further features in the described preferred embodiments the vital signs measuring unit is integrated with or mounted on the portable detection system.

According to still further features in the described preferred embodiments the vital signs measuring unit is designed and configured to generate a signal to the activation unit to activate the portable detection system when the vital signs of the mammal meet a predetermined set of criteria.

According to still further features in the described preferred embodiments the kit further comprises an atmospheric condition measuring unit for measuring at least one atmospheric condition.

According to still further features in the described preferred embodiments the atmospheric condition measuring unit is integrated with or mounted on the portable detection system.

According to still further features in the described preferred embodiments sampling device is reusable.

According to still further features in the described preferred embodiments sampling device is adapted to sample fluids.

According to still further features in the described preferred embodiments sampling device is adapted to sample solids.

According to still further features in the described preferred embodiments sampling device comprises a syringe and a syringe needle.

According to still further features in the described preferred embodiments the sampling device is adapted to continuously sample the environmental materials.

According to still further features in the described preferred embodiments the treating elements are selected from the group consisting of a filter, an enriching unit, an elution unit, a heating unit, an irradiation unit, a labeling unit, a separating column and a sorter.

According to still further features in the described preferred embodiments the treating elements comprise at least one biological material.

According to still further features in the described preferred embodiments the sampling device comprises a plurality of containers and treating elements for treating fluids in the plurality of containers prior to the transfer of the fluids to the sensing device.

According to still further features in the described preferred embodiments the at least one treating element is designed and constructed to perform a nucleic acid amplification procedure.

According to still further features in the described preferred embodiments at least one of the plurality of containers is kept at a predetermined pH level at all times.

According to still further features in the described preferred embodiments the kit further comprises a medical injection device, for injecting at least one medicament to a mammal.

According to still further features in the described preferred embodiments the kit further comprises at least one medicament.

According to still further features in the described preferred embodiments the injection device comprises: an injection needle; a plurality of syringes, each having a plunger slidably disposed therein; a driving mechanism having a plurality of drive members, each drive member of the plurality of drive members being operable to engage a plunger of a respective syringe of the plurality of syringes; and a redirector for redirecting medicaments, released by at least one syringe of the plurality of syringes, into the injection needle.

According to still further features in the described preferred embodiments the injection device further comprises a second communication unit, operable to communicate with the portable detection system and/or the communication network, and further wherein the driving mechanism is designed and constructed to receive activation signals from the second communication unit, to thereby activate the at least one syringe.

According to still further features in the described preferred embodiments the injection device further comprises a user display.

According to still further features in the described preferred embodiments the injection device further comprises an audio device.

According to still further features in the described preferred embodiments the plurality of reaction chambers are configured so as to enable sustaining a under-pressure environment within the plurality of reaction chambers.

According to still further features in the described preferred embodiments the plurality of reaction chambers are configured such that the sensor does not substantially obstruct fluid flow in and out of the plurality of reaction chambers.

According to still further features in the described preferred embodiments the plurality of channels are connected to the plurality of reaction chamber substantially above a bottom surface thereof.

According to still further features in the described preferred embodiments a body of the sensing device is capable of allowing transmission of light having a predetermined wavelength therethrough.

According to still further features in the described preferred embodiments the body comprises a material selected from the group consisting of silicon, plastic and glass.

According to still further features in the described preferred embodiments the portable detection system further comprises a temperature control unit for controlling a temperature of the sensing device, the planar light detector and/or the light source.

According to still further features in the described preferred embodiments the portable detection system further comprises at least one selective filter positioned between the sensing device and the planar light detector, the at least one selective filter being capable of transmitting the optical signals and preventing transmission of the excitation light.

According to still further features in the described preferred embodiments the portable detection system further comprises an optical focusing device for focusing the optical signal on the planar light detector.

According to still further features in the described preferred embodiments the optical focusing device comprises a plurality of lenses positioned to substantially prevent cross talks between different optical signals of different sensors.

According to still further features in the described preferred embodiments the portable detection system further comprises an electronic circuitry designed and constructed for controlling the plurality of micro-pumps.

According to still further features in the described preferred embodiments the electronic circuitry comprises at least one feedback line for monitoring operation and/or status of the plurality of micro-pumps.

According to still further features in the described preferred embodiments the transport mechanism comprises a pumping device, capable of generating a under-pressure in the plurality of reaction chambers and the plurality of fluid channels.

According to still further features in the described preferred embodiments the transport mechanism further comprises a vacuum chamber connected to the pumping device and capable of maintaining a under-pressure environment.

According to still further features in the described preferred embodiments the transport mechanism further comprises a pressure sensor for sensing a pressure at an inlet of the vacuum chamber.

According to still further features in the described preferred embodiments the transport mechanism further comprises a flow sensor for sensing flow parameters of the sample fluid.

According to still further features in the described preferred embodiments the transport mechanism further comprises at least one tap for controlling the flow parameters.

According to still further features in the described preferred embodiments the transport mechanism further comprises at least one valve for activating and deactivating the transport of the sample fluid.

According to still further features in the described preferred embodiments the transport mechanism further comprises a hydrophobic filter for protecting at least one component of the transport mechanism.

According to still further features in the described preferred embodiments the portable detection system further comprises electronic circuitry for controlling flow rate of the sample fluid.

According to still further features in the described preferred embodiments the electronic circuitry is designed and constructed to allow equal filling of the sample fluid in the plurality of reaction chambers.

According to still further features in the described preferred embodiments the kit further comprises a storage unit for storing the sensing device, the sampling device and/or the fluids, and maintaining a predetermined temperature thereof.

According to still further features in the described preferred embodiments the storage unit comprises: at least one cavity having a plurality of temperature control units and configured to receive a plurality of objects in a manner such that a plurality of thermal communications are established between the plurality of temperature control units and the plurality of objects, each of the plurality of objects being selected from the group consisting of the sensing device, the sampling device and the fluids; and a controller, configured to receive temperature information from the plurality of temperature control units and to independently activate each temperature control unit to maintain a predetermined temperature of a respective object.

According to still further features in the described preferred embodiments the storage unit further comprises a portable power source for supplying energy to the plurality of temperature control units, wherein the controller is configured to receive power level information from the power source.

According to still further features in the described preferred embodiments the storage unit further comprises an arrangement of presence sensors, communicating with the controller, for determining a number and position of objects occupying the storage unit at all times, wherein the controller is configured to independently activate and deactivate each temperature control unit, based on presence information received from the arrangement of presence sensors.

According to still further features in the described preferred embodiments the storage unit further comprises a display, communicating with the controller, for displaying temperature, operation status and/or presence information transmitted by the controller.

According to still further features in the described preferred embodiments the storage unit further comprises a communication unit for connecting the storage unit to a communication network.

According to yet another aspect of the present invention there is provided a method of distributing information of a presence of agents in the environment, the method comprising: sampling the environment thereby providing a sample; delivering the sample through a plurality of channels to thereby contact the sample with at least one sensor, capable of generating detectable signals when exposed to the agents; upon a detection of the detectable signals, processing the detectable signals so as to determine presence or level of an agent in the sample; and transmitting signals indicative of the presence or level of the agent over a communication network.

According to further features in preferred embodiments of the invention described below, the method further comprises determining a location at which the sampling is performed and transmitting the location over the communication network.

According to still further features in the described preferred embodiments the method further comprises generating at least one sensible signal being indicative of presence, level or absence of harmful agents.

According to still further features in the described preferred embodiments the method further comprises continuously monitoring environmental conditions, wherein the sampling is performed when the environmental conditions meet a predetermined set of criteria.

According to still further features in the described preferred embodiments the method further comprises transmitting an identification code over the communication network.

According to still further features in the described preferred embodiments the method further comprises detection presence of agents in the environment by a process selected from the group consisting of optical detection, gas chromatography, mass spectroscopy, time of flight analysis and any combination thereof.

According to still further features in the described preferred embodiments the method further comprises monitoring vital signs of a mammal identified as being possibly in contact with the agent and transmitting the vital signs over the communication network.

According to still further features in the described preferred embodiments the method further comprises monitoring atmospheric condition and transmitting the atmospheric condition over the communication network.

According to still further features in the described preferred embodiments the method further comprises injecting at least one medicament to a mammal identified as being possibly in contact with the agent.

According to still further features in the described preferred embodiments the contact of the sample with the at least one sensor is at a predetermined pH level.

According to still further features in the described preferred embodiments the method further comprises capturing an image of at least a portion of the environment and capturing and transmitting the image over the communication network.

According to still further features in the described preferred embodiments the method further comprises subjecting the sample to a nucleic acid amplification procedure.

According to still further features in the described preferred embodiments the method further comprises detecting a shape of agents present in the sample.

According to still further features in the described preferred embodiments the method further comprises further comprises detecting motion characteristics of agents present in the sample.

According to still further features in the described preferred embodiments the detectable signals comprise optical signals.

According to still further features in the described preferred embodiments the method further comprises controlling a temperature of the sensor and the sample.

According to still further features in the described preferred embodiments the method further comprises binding components of the gas sample to a liquid phase.

According to still further features in the described preferred embodiments the method further comprises binding components of the solid sample to a liquid phase.

According to still another aspect of the present invention there is provided a medical injection device, comprising: an injection needle; a plurality of syringes, each having a plunger slidably disposed therein; a driving mechanism having a plurality of drive members, each drive member of the plurality of drive members being operable to engage a plunger of a respective syringe of the plurality of syringes; a redirector for redirecting medicaments, released by at least one syringe of the plurality of syringes, into the injection needle.

According to further features in preferred embodiments of the invention described below, the device further comprises a communication unit, operable to communicate a communication network, wherein the driving mechanism is designed and constructed to receive activation signals from the communication unit, to thereby activate the at least one syringe.

According to still further features in the described preferred embodiments the device further comprises a user display.

According to still further features in the described preferred embodiments the device further comprises an audio device.

According to an additional aspect of the present invention there is provided a distributed detection system for detection of the presence of harmful agents in an environment, comprising a central monitoring unit and a plurality of portable agent detection systems being mounted on mobile vectors for release into the environment, each portable agent detection system of the plurality of portable agent detection systems comprising: a sensing device having a substrate formed with a plurality of reaction chambers and a plurality of channels interconnecting at least a portion of the plurality of reaction chambers, wherein at least a portion of the plurality of reaction chambers comprises a sensor, capable of generating a detectable signal when exposed to the agents; a detector capable of receiving signals from the sensing device and providing an image of sensors generating the optical signals; and a communication unit, for connecting the portable system to a communication network.

According to further features in preferred embodiments of the invention described below, the communication unit is designed to transmit signals representing presence, level or absence of the agents.

According to still further features in the described preferred embodiments the portable detection system further comprises a positioning unit, for determining a location of the system, wherein the communication unit is designed to transmit the location over the communication network.

According to still further features in the described preferred embodiments the portable detection system further comprises a personal accessory device, wherein the sensing device, the detector and the communication unit are integrated with or installed in a body of the personal accessory device.

According to still further features in the described preferred embodiments the personal accessory device is selected from the group consisting of a cellular telephone, a laptop and a personal digital assistant.

According to still further features in the described preferred embodiments the portable detection system further comprises an activation unit for activating or selecting an operational mode of the system.

According to still further features in the described preferred embodiments the portable detection system further comprises a user interface, wherein the activation unit is controllable by the user interface.

According to still further features in the described preferred embodiments the user interface is designed and configured to generate at least one sensible signal being indicative of presence, level or absence of harmful agents.

According to still further features in the described preferred embodiments a level of the at least one sensible signal is selected to allow sensation of the at least one sensible signal at large distances.

According to still further features in the described preferred embodiments the user interface is designed and configured to generate at least one sensible signal being indicative of an operative status and/or impermeability level of the system.

According to still further features in the described preferred embodiments the activation unit is in communication with a central location and being controllable thereby, hence allowing a remote activation or operational mode selection of the system.

According to still further features in the described preferred embodiments the portable detection system further comprises a user display.

According to still further features in the described preferred embodiments the user display is in communication with a central location and being controllable thereby, hence allowing the central location to communicate, at least unilaterally, with a user.

According to still further features in the described preferred embodiments the portable detection system further comprises a supplementary sensing unit capable of continuously monitoring environmental conditions, and generating a signal to the activation unit to activate the system when the environmental conditions meet a predetermined set of criteria.

According to still further features in the described preferred embodiments the predetermined set of criteria comprises preliminary detection of a potentially harmful agent.

According to still further features in the described preferred embodiments the portable detection system further comprises detection hardware.

According to still further features in the described preferred embodiments the detection hardware is selected from the group consisting of optical detection hardware, a gas chromatograph, a gas chromatograph-mass spectrometer, a time of flight analyzer and any combination thereof.

According to still further features in the described preferred embodiments the portable detection system further comprises vital signs measuring unit for measuring vital signs of a mammal carrying the system.

According to still further features in the described preferred embodiments the vital signs measuring unit is designed and configured to generate a signal to the activation unit to activate the system when the vital signs of the mammal meet a predetermined set of criteria.

According to still further features in the described preferred embodiments the portable detection system further comprises an atmospheric condition measuring unit for measuring at least one atmospheric condition.

According to still further features in the described preferred embodiments at least one of the plurality of reaction chambers is kept at a predetermined pH level at all times.

According to still further features in the described preferred embodiments the portable detection system further comprises an armored encapsulation.

According to still further features in the described preferred embodiments the armored encapsulation is at least partially impermeable and capable of withstanding extreme thermal and/or mechanical conditions.

According to still further features in the described preferred embodiments the portable detection system further comprises an image capturing unit.

According to still further features in the described preferred embodiments the portable detection system further comprises an input-output audio unit.

According to still further features in the described preferred embodiments the communication unit is supplemented with at least one communication protocol, tangibly embodied in a readable memory, the at least one communication protocol being configured to allow a takeover of the communication network.

According to still further features in the described preferred embodiments the portable detection system further comprises a nucleic acid amplification unit.

According to still further features in the described preferred embodiments the portable detection system further comprises a shape detector.

According to still further features in the described preferred embodiments the portable detection system further comprises a motion detector.

According to still further features in the described preferred embodiments the detectable signal comprises optical signal.

According to still further features in the described preferred embodiments the portable detection system further comprises a data processor, supplemented by an algorithm for receiving image information from the detector and determining presence or level of the agents.

According to still further features in the described preferred embodiments the portable detection system further comprises a control unit for sending control signals to the sensing device.

According to still further features in the described preferred embodiments the portable detection system further comprises a temperature control unit for controlling a temperature of the sensing device and/or the detector.

According to still further features in the described preferred embodiments the temperature control unit is selected from the group consisting of a thermoelectric device, a liquid cooler, a gas cooler and a blower.

According to still further features in the described preferred embodiments the algorithm is capable of determining concentration of the agents.

According to still further features in the described preferred embodiments the sensing device is removable.

According to still further features in the described preferred embodiments the sensing device is disposable.

According to still further features in the described preferred embodiments at least a portion of the plurality of reaction chambers are sequentially interconnected via at least a portion of the channels.

According to still further features in the described preferred embodiments the sensor is a biological sensor.

According to still further features in the described preferred embodiments the biological sensors is capable of producing a bioluminescent material.

According to still further features in the described preferred embodiments the biological sensors is capable of producing a phosphorescent material.

According to still further features in the described preferred embodiments the biological sensor is capable producing a fluorescent material.

According to still further features in the described preferred embodiments the planar light detector comprises a matrix having a plurality of addressable elementary units, each being capable of converting the optical signal into an electrical signal.

According to still further features in the described preferred embodiments the elementary units of the planar light detector are selected from the group consisting of positive-intrinsic-negative photodiodes, avalanche photodiodes, silicon chips and photomultipliers.

According to still further features in the described preferred embodiments the portable detection system further comprises a light source for emitting excitation light so as to excite the sensor to thereby emit the optical signal.

According to still further features in the described preferred embodiments the light source comprises a light emitting diode.

According to still further features in the described preferred embodiments the light emitting diode is coupled to a collimator capable of redirecting the excitation light to form a substantially collimated light beam.

According to still further features in the described preferred embodiments the light source comprises an arrangement of light emitting diodes.

According to still further features in the described preferred embodiments each light emitting diode of the arrangement of light emitting diodes is coupled to a collimator capable of redirecting the excitation light to form a substantially collimated light beam.

According to still further features in the described preferred embodiments the sensing device comprises a plurality of waveguides designed and constructed to distribute the excitation light among the plurality of chambers in a manner such that impingement of the excitation light on the biological sensor is maximized and impingement of the excitation light on a surface of the substrate is minimized.

According to still further features in the described preferred embodiments the portable detection system further comprises a transport mechanism for actuating transport of a sample fluid in the plurality of fluid channels, thereby to fill the plurality of reaction chambers with the sample fluid.

According to still further features in the described preferred embodiments the portable detection system further comprises a draining system and further wherein the transport mechanism is capable of maintaining a continues flow of the sample fluid in the plurality of fluid channels thereby to continuously replace the sample fluid in the plurality of reaction chambers.

According to still further features in the described preferred embodiments the detector is capable of providing the image substantially in real time.

According to still further features in the described preferred embodiments a portion of the plurality of reaction chambers comprises a material capable of generating a detectable reference signal at all times.

According to still further features in the described preferred embodiments the pumping device comprises a plurality of micro-pumps.

According to still further features in the described preferred embodiments the transport mechanism comprises an electric field generator, for generating a non-uniform electric field capable of inducing polarization on molecules of the sample fluid, hence to fill the plurality of reaction chambers with the sample fluid via dielectrophoresis.

According to still further features in the described preferred embodiments the plurality of fluid channels are designed and constructed such that fluid sample flows therethrough via capillary forces.

According to still further features in the described preferred embodiments the sample is a liquid sample.

According to still further features in the described preferred embodiments the portable detection system further comprises a mechanism for binding components of the gas sample to a liquid phase.

According to still further features in the described preferred embodiments the portable detection system further comprises a mechanism for binding components of the solid sample to a liquid phase.

According to still further features in the described preferred embodiments the biological sensors comprises a population of cells, the population of cells including a reporter expression construct being expressible in a cell of the population when exposed to the agent.

According to still further features in the described preferred embodiments the population of cells is eukaryotic cells.

According to still further features in the described preferred embodiments the population of cells is prokaryotic cells.

According to still further features in the described preferred embodiments each of the reporter expression construct includes a cis-acting regulatory element being operably fused to a reporter gene.

According to still further features in the described preferred embodiments the reporter gene is selected from a group consisting of a fluorescent protein, an enzyme and an affinity tag.

According to still further features in the described preferred embodiments the cis-acting regulatory element is a promoter.

According to still further features in the described preferred embodiments the promoter is selected from the group consisting of MipA, LacZ, GrpE, Fiu, MalPQ, oraA, nhoA, recA, oIsAB and yciD.

According to still further features in the described preferred embodiments the cis-acting regulatory element is stress regulated.

According to still further features in the described preferred embodiments the agent is selected from the group consisting of a condition and a substance.

According to still further features in the described preferred embodiments the condition is selected from the group consisting of a temperature condition and a radiation condition.

According to still further features in the described preferred embodiments the substance is a naturally occurring product or a synthetic product.

According to still further features in the described preferred embodiments the populations of cells is tagged.

According to yet an additional aspect of the present invention there is provided a storage unit, comprising: at least one cavity having a plurality of temperature control units and configured to receive a plurality of objects in a manner such that a plurality of thermal communications are established between the plurality of temperature control units and the plurality of objects; and a controller, configured to receive temperature information from the plurality of temperature control units and to independently activate each temperature control unit to maintain a predetermined temperature of a respective object.

According to further features in preferred embodiments of the invention described below, each of the plurality of objects is selected from the group consisting of a sensing device, a sampling device and a collected sample.

According to still further features in the described preferred embodiments the controller comprises an internal power source.

According to still further features in the described preferred embodiments the storage unit further comprises a portable power source for supplying energy to the plurality of temperature control units, wherein the controller is configured to receive power level information from the power source.

According to still further features in the described preferred embodiments each of the plurality of temperature control units comprises a cooler which is selected from the group consisting of a thermoelectric device, a liquid cooler, a gas cooler and a blower.

According to still further features in the described preferred embodiments each of the plurality of temperature control units comprises a temperature sensor.

According to still further features in the described preferred embodiments the controller is configured to receive operation status information from the temperature control units.

According to still further features in the described preferred embodiments the storage unit further comprises an arrangement of presence sensors, communicating with the controller, for determining a number and position of sensing devices occupying the storage unit at all times, wherein the controller is configured to independently activate and deactivate each temperature control unit, based on presence information received from the arrangement of presence sensors.

According to still further features in the described preferred embodiments the storage unit further comprises a display, communicating with the controller, for displaying temperature, operation status and/or presence information transmitted by the controller.

According to still further features in the described preferred embodiments the storage unit further comprises a communication unit for connecting the storage unit to a communication network.

According to still further features in the described preferred embodiments the controller is configured to transmit temperature, operation status and/or presence information via the communication unit over the communication network.

The present invention successfully addresses the shortcomings of the presently known configurations by providing method, system and kit for detecting and warning presence of chemical, biological or radioactive agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is schematic illustration of portable system for detecting agents present in a sample, according to a preferred embodiment of the present invention.

FIG. 1b is schematic illustration of a sensing device employed by the system, according to a preferred embodiment of the present invention.

FIG. 2a-b are schematic illustrations of the reaction chambers and the fluid channels of the sensing device in a preferred embodiment in which the chambers and the channels are arranged in one or more sequential arrays.

FIG. 3a is a schematic illustration of the sensing device in the preferred embodiment in which a plurality of micro-pumps is employed.

FIG. 3b is a schematic illustration of one micro-pump, according to a preferred embodiment of the present invention.

FIG. 3c is a schematic diagram exemplifying a configuration of an electronic circuitry controlling the micro-pump of the sensing device shown in FIG. 3a, according to a preferred embodiment of the present invention.

FIG. 4 is a schematic illustration of a transport mechanism in a preferred embodiment in which external pumping is utilized, according to a preferred embodiment of the present invention.

FIG. 5 is a schematic illustration of a portion of the sensing device in which the transport mechanism comprises an electric field generator, according to a preferred embodiment of the present invention.

FIG. 6 is a schematic illustration of a portion of the device in a preferred embodiment in which the transport of the sample fluid is generated by hydrostatic pressure.

FIG. 7 is a schematic illustration of a fluid channel in a preferred embodiment in which the sample fluid flows via capillary forces.

FIG. 8 is a schematic illustration of a logical and physical division of the sensing device, according to the presently preferred embodiment of the invention.

FIG. 9 is a simplified illustration of a light detector, according to a preferred embodiment of the present invention.

FIGS. 10a-d are schematic illustrations of the sensing device in a preferred embodiment in which spatial separation of the excitation light from the optical signal is employed, using a plurality of waveguides.

FIG. 11a is a schematic illustration of a side view of a light source, in a preferred embodiment in which waveguides are employed.

FIG. 11b is a schematic illustration of a top view of the light beam outputted by the light source, according to a preferred embodiment of the present invention.

FIG. 12 is a schematic illustration of the system in a preferred embodiment in which the device is positioned between the light source and the light detector.

FIG. 13 is a schematic illustration of the system in a preferred embodiment in which a plurality of external optical fibers is employed.

FIG. 14 is a schematic illustration of the light path of the excitation light once entering the reaction chamber, according to a preferred embodiment of the present invention (FIG. 14 is rotated anticlockwise by 90° relative to FIG. 13).

FIGS. 15a-c are schematic illustrations of a light source of the system, according to a preferred embodiment of the present invention.

FIG. 16a is a schematic illustration of the light source in a preferred embodiment in which the light source is an arrangement of light emitting devices.

FIGS. 16b-c are schematic illustrations of the system in a preferred embodiment in which the light source of FIG. 17a is positioned between the device and the light detector.

FIG. 17 is a schematic illustration of a storage unit for storing the sensing device, according to a preferred embodiment of the present invention.

FIG. 18 is a flowchart diagram of a method distributing information of a presence of agents in the environment, according to a preferred embodiment of the present invention.

FIG. 19a is a schematic illustration of a kit for detecting agents present in a sample, according to a preferred embodiment of the present invention.

FIG. 19b is a schematic illustration of a medical injection device, according to a preferred embodiment of the present invention.

FIG. 19c is a schematic illustration of a sampling device, according to according to a preferred embodiment of the present invention.

FIGS. 20a-c are flowchart diagrams of several general treatment scenarios, which can be employed on sampled materials according to a preferred embodiment of the present invention.

FIGS. 21a-c are flowchart diagrams of treatment scenarios for sampling of air (FIG. 21a), liquid (FIG. 21b) and surface (FIG. 21c), according to a preferred embodiment of the present invention.

FIGS. 22a-b are electronic diagrams of a CMOS detector, which can be used as a light detector, according to a preferred embodiment of the present invention.

FIG. 23a is a schematic illustration of the slicing technique of the method of FIG. 21, according to a preferred embodiment of the present invention.

FIG. 23b is a schematic illustration of an equivalent light emitter which can be used in the slicing technique of the method of FIG. 21, according to a preferred embodiment of the present invention.

FIG. 23c is a schematic illustration of the spreading of an optical signal through an aperture of the reaction chamber, according to a preferred embodiment of the present invention.

FIG. 24 is a schematic calculation diagram which can be implemented for the calculation of optical coefficients, according to a preferred embodiment of the present invention.

FIG. 25 illustrates light propagation from the equivalent light emitter to a lens, according to a preferred embodiment of the present invention.

FIG. 26 illustrates the scattering solid angle of the emitted light rays, according to a preferred embodiment of the present invention.

FIGS. 27a-d show results of an experiment performed with fresh biological sensors.

### DESCRIPTION OF THE PREFERRED EMBODIMENTS

The present invention is of a method, system and kit for providing early detection and warning of presence of harmful agents, such as, but not limited to, chemical, biological or radioactive agents. Specifically, the present invention can be used to measure and analyze water, air or surface toxicity using wetware or hardware, and distribute detection information over a communication network. The present invention is further of a medical injection device which can be used in case of injury or exposure to a harmful agent.

The principles and operation of a device, apparatus and system for detecting and/or identifying agents according to the present invention may be better understood with reference to the drawings and accompanying descriptions.

Before explaining at least one embodiment of the invention in detail, it is to be understood that the invention is not limited in its application to the details of construction and the arrangement of the components set forth in the following description, illustrated in the drawings or exemplified by the Examples. The invention is capable of other embodiments or of being practiced or carried out in various ways. Also, it is to be understood that the phraseology and terminology employed herein is for the purpose of description and should not be regarded as limiting.

According to one aspect of the present invention, there is provided a system 10 for detecting one or more agents.

As used herein the term “agent” generally refers to a biological, chemical or radioactive material, in any form, including, without limitation fluid (gas or liquid), solid (particulates, aggregates or contaminated surfaces), vapor, aerosol and the like. The agent can be a molecule or a mixture of molecules in a liquid, gaseous, aerosol or solid medium. When the agent is in liquid medium, it may be in solution or carried by the liquid medium. When the agent is in a solid medium is may be absorbed or adsorbed in the solid medium.

Examples of agents include, but are not limited to, small molecules such as naturally occurring compounds (e.g., compounds derived from plant extracts, microbial broths, and the like) or synthetic compounds having molecular weights of less than about 10,000 daltons, preferably less than about 5,000 daltons, and most preferably less than about 1,500 daltons, electrolytes, metals, peptides, nucleotides, saccharides, fatty acids, steroids and the like.

As used herein the term “about” refers to ±10%.

Agents typically, but not obligatory, include at least one functional group necessary for biological interactions (e.g., amine group, carbonyl group, hydroxyl group, carboxyl group).

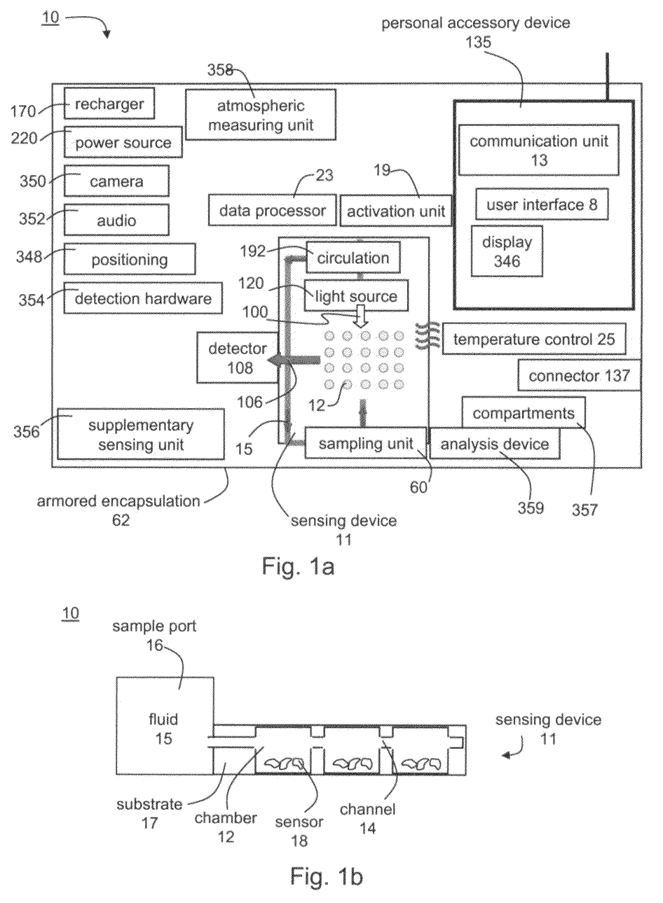

Referring now to the drawings, FIG. 1a is a schematic illustration of system 10, according to a preferred embodiment of the present invention. System 10 preferably comprises a sensing device 11, a detector 108 and a communication unit 13.

Sensing device 11 is better illustrated in FIG. 1b. Hence, device 11 preferably has a plurality of reaction chambers 12 and a plurality of channels 14 interconnecting at least a portion of reaction chambers 12. Reaction chambers 12 and channels 14 can be formed in or integrated with a substrate 17 in any fabrication method known in the art. Sensing device 11 is preferably environmentally sealed. Additionally, device 11 is preferably disposable and can be removed from system 10, e.g., for replacing device 11 with another device or for storage. Thus, substrate 17 preferably comprises a disposable material, such as, but not limited to, silicon, plastic and glass.

Reaction chamber 12 preferably comprises a sensor 18, capable of generating a detectable signal when exposed to the agents. Many types of sensors are contemplated, for example, wetware sensors (e.g., arrays of immobilized reporter cells) or hardware based detectors (e.g., electromagnetic radiation emitters, agent-sensitive semiconductors). Various types of sensors which are suitable for the present embodiments are found in Franz L. Dickert et al., “Sensor strategies for microorganism detection—from physical principles to imprinting procedures,” Anal Bioanal Chem (2003) 377:540-549, and U.S. Pat. Nos. 6,743,581, 6,710,711, 6,679,099, 6,558,626, 6,448,064, 6,197,503 and 6,159,681, the contents of which are hereby incorporated by reference.

The detectable signals are detected by detector 108 which generates a detection event signal. According to a preferred embodiment of the present invention any detectable signal can be generated by sensors 18, depending on the type of sensor being utilized. Representative examples of detectable signals include, without limitation, optical, electrical and electrochemical signals. The type of detector 108 is thus selected according to the type of signals generated by sensors 18. Hence, detector 108 can be, for example, a planar light detector, a sound acoustic wave resonator, a surface plasmon resonance detector and the like. According to a preferred embodiment of the present invention system 10 further comprising a data processor 23, for processing signals received from detector 108 hence being in data communication therewith.

When the detectable signals are optical, substrate 17 is preferably made transparent to the optical signals. Substrate 17 can also be manufactured so as to selectively allow transmission of light having a predetermined wavelength. This can be done, for example, by doping substrate 17 by an impurity, whereby the type and concentration of the impurity is selected in accordance with the wavelength of the optical signals.

Communication unit 13 serves for transmitting signals over a communication network, such as, but not limited to, wireless local area network (WLAN), Wi-Fi® network, Bluetooth® network, cellular network and the like. Preferably, communication unit 13 is configured to allow connection to more that one communication network. This embodiment is particularly useful when system 10 operates in districts in which different regions are covered by different communication network. According to a preferred embodiment of the present invention the signals transmitted by communication unit 13 represent detection information (e.g., presence, level or absence of harmful agents). To prevent unauthorized parties from monitoring or intervening with the data transfer, communication unit 13 is preferably configured to transmit and receive encoded information. The encoding of the information, prior to the transmission over the network and the decoding of the received signals can be using an encryption algorithm employed by data processor 23. Many encryption algorithms are contemplated, including, without limitation, an RSA algorithm, found, e.g., in an article by Ronald L. Rivest, Adi Shamir, and Leonard M. Adleman, entitled “A Method for Obtaining Digital Signatures and Public-Key Cryptosystems,” published in Communications of the ACM, 21, 2:120-126, (1978).

Optionally and preferably, system 10 further comprises a positioning unit 9 to allow system 10 to determine its location and, optionally, transmit location data using communication unit 13. Many positioning technologies are contemplated. Representative examples include, without limitation global positioning systems, commonly known as GPS, network based positioning, in which the location of the detector is computed by triangulation of its signal between transmission towers, motion-based positioning in which the location is calculated based on the motion parameters of the detector, and cell-identification in which the environment is divided into a plurality of geometric elements and each entry of the detector into a respective element is monitored and recorded.

System 10 is preferably provided with one or more connectors 137 so as to allow information transfer with other devices, such as, but not limited to, a personal accessory device (cellular telephone, laptop, personal digital assistant, etc.). Alternatively or additionally, a personal accessory device 135 (e.g., a cellular telephone) can be integrated with or installed in system 10. In this embodiment, communication unit 13, user interface 8 and display 346 can be integrated in device 135.

According to a preferred embodiment of the present invention system 10 has a prolonged shelf life, so as to allow accumulation of large number of items of system 10 to be distributed to the population. Preferably, system 10 is designed to be stored in a non-operative mode under ambient conditions for prolonged periods of time without loss of functionality. Selected components of system 10, (e.g., device 11 including sensors 18) may be stored under lower temperatures (e.g., 0-10° C.) to further extend the shelf life of system 10.

According to a preferred embodiment of the present invention device 11 and detector 108 are designed and constructed to provide detection information within predetermined and variable time periods. Specifically, the time frame for detecting a particular agent dependent upon the magnitude of its threat. For example, for acute, life threatening agents, the detection time is preferably from a few seconds up to one or a few minutes; for less harmful agents, the detection can be within several (say about 15) the detection is preferably within. Additionally, system 10 is preferably capable of providing preliminary detection information at an early time (say within a few seconds), and a final detection information at a later time, depending on the type of threat and detection means.

As used herein the term “about” refers to ±10%.

Device 11 further comprises one or more sample ports 16, being in fluid communication with chambers 12 via channels 14. Sample ports 16 serve for feeding sample fluid 15 (gas or liquid) into channels 14 through which sample fluid 15 flows into reaction chambers 12 and interacts with the sensors. When the agent is in gaseous or solid state, its components (e.g., organic components) are preferably bound to a liquid phase prior to the feeding of sample port 16.

Given that a sufficient number of sensors are utilized, the intensity of the signal, generated by sensors 18, typically depends on the amount of detectable agent being in contact therewith. For a given sample fluid having a given concentration, the intensity of the signal is proportional to the amount of sample fluid present in reaction chambers 12. To optimize correlation between the intensity of the signal and the concentration of the agent in sample fluid 15, reaction chambers 12 are preferably of substantially equal volume. The advantage of this embodiment is that when chambers 12 are filled, the occupation ratio between the agent and sensors 18 does not vary from one chamber to another, so that, once a functional relationship between the intensity of the signal and the concentration of agent is established (e.g., using a simple calibration curve, a mathematical model, etc.), the same functional relationship can be used for many reaction chambers.

Reaction chambers 12 are preferably addressable so as to allow imaging thereof, as further detailed hereinunder. In this embodiment, at least a few of reaction chambers 12 may comprise different sensors, each capable of generating the detectable signal when exposed to a different agent.

System 10 is preferably compact and manufactured in mass production, so as to allow distribution to many individuals. Hence, device 11 is typically of small dimensions. Preferably, the area of device 11 is less than about 10 cm², more preferably less than 1 cm², most preferably less than 0.1 cm². The number of reaction chambers and fluid channels of system 10 is not limited, and may vary from a few to a few hundreds of thousands of reaction chambers positioned on the same device. Thus, device 11 is preferably a microfluidic device so as to facilitate forming therein or integrating therewith a large number of reaction chambers and fluid channels. When reaction chambers 12 comprise different sensors, device 11 is capable of providing a multiplexed detection of an enormous amount of different agents.

Fluid channels 14 are preferably microfluidic channels. Transport of sample 15 from sample port 16 through channels 14 and into reaction chambers 12 can be effected using a variety of methods which are known in the art. Preferably sample transport is effected in a manner which enables provision of an equal fluid volume to each of reaction chambers 12. This may be done by a judicious positioning of fluid channels 14 and reaction chambers 12, as further detailed hereinunder.

Before providing a further detailed description of system 10, as delineated hereinabove and in accordance with the present embodiment, attention will be given to the potential applications offered thereby.

Hence, a particular feature of system 10, besides being compact and easy to carry by the population, is its ability to communicate over the communication network thereby to distribute information. As will be appreciated by one ordinarily skilled in the art, the portability use of many portable systems which communicate over the network and interchange detection and, optionally, location data can be used to detect presence, level and location of a threat and to distribute the information, substantially in real-time, both to the appropriate authorities and among the population.

Thus, detection systems, manufactured in accordance with preferred embodiments of the present invention, can be released to the environment by placing the detection systems on vectors in the environment.

As used herein, a “vector” refers to an entity having a self-relocating ability. For example, a “vector” can be a civilian, a law-enforcement officer, a vehicle, an animal and the like.

The detection systems can also be integrated with or mounted on personal accessories, such as, but not limited to, cellular telephones, personal digital assistants, laptops and key holders, which are typically carried by the residents on normal routine. In this embodiment, the vectors are individuals of the population, which, by following their daily routines, span a substantially large detection area.

The activation of the nearby detectors can be done either by instructing the respective individuals which carry the nearby detectors to locally activate the detectors, or by performing a remote activation at the central location. Additionally, certain individuals can be alerted of the threat and instructed to take the necessary precautions.

Moreover, the use of a plurality of portable and communicative detection systems allows identification of clustering of detection events by combining detection and location information received from various locations. The clustering can serve both as confirmation for the presence, level and location of the harmful agent, and to assess the diffusion rate, e.g., by repeating the clustering identification at different instants of time.

Thus, system 10 has the advantage of providing early warning of a potential unconventional attack, such as biological, chemical or radioactive attack. System 10 is effective against deliberate events (e.g., biological, chemical or radioactive terrorism), accidents causing release of harmful agents (e.g., cargo spills) and natural events (e.g., epidemics). As will be appreciated by one of ordinary skill in the art, system 10 provides practical, inexpensive and easily applicable solution to the problem of harmful agent detection.

Referring now again to the FIG. 1, system 10 may further comprise other means of detection, e.g., detection hardware 354, which can be any detection hardware known in the art, such as, but not limited to, an optical detection device (e.g., a laser device), a gas chromatograph, a gas chromatograph-mass spectrometer, a time of flight analyzer and any combination thereof. Detection hardware 354, is preferably used for short range detection, for example, by emitting an optical signal toward the agent and receiving optical response therefrom. This embodiment is particularly useful for detecting specific toxicants, such as, but not limited to, airborne and/or surface-laden lethal toxic particles, e.g., anthrax and chemical agents. Additionally, detection hardware 354 can be used to provide data regarding air-flow directions.

System 10 preferably comprises a power source 220 for supplying energy to its components, e.g., processing unit 36 and other components which may be employed, as further detailed hereinunder. Power source 220 is preferably portable, and can be replaceable or rechargeable, integrated with, or being an accessory to system 10. Representative examples include, without limitation a solar power source, a mobile a voltage generator, an electrochemical cell, a traditional secondary (rechargeable) battery, a double layer capacitor, an electrostatic capacitor, an electrochemical capacitor, a thin-film battery (e.g., a lithium cell), a microscopic battery and the like. In embodiments in which Power source 220 is rechargeable, system 10 preferably comprises a recharger 170, which can be integrated with or supplied separately to system 10 as desired.

Alternatively, power source 220 can be a fixed power source, for example, a power source from a wall socket or a fixed voltage generator. According to a preferred embodiment of the present invention power source 220 can be disconnected from system 10 without the need to open sealed components (e.g., cassette 34).

The type and size of power source 220 as well as the amount of energy stored therein may vary, depending on the required power and, in some embodiments, on the component in which power source 220 is implemented. Preferably, the life time of power source 220 when system 10 is not operative is comparable to the shelf life of system 10. In operative mode, the life time of power source 220 is preferably above 1 hour more preferably above 10 hours most preferably above 100 hours. System 10 can also comprise a backup power source, which can be used to backup power source 220 in case its power level is to low and/or during the replacement of power source 220.

System 30 preferably comprises an activation unit 19 for activating or selecting an operational mode of the portable agent detector. Activation unit 19 can be controlled by a user, for example, through a user interface 8. Being primarily intended to be distributed to the general population, system 10, and particularly user interface 8 is preferably “user friendly.” This can be achieved by designing user interface 8 in a manner such that the number of actions which are required from the user to guaranty full functionality of the unit is minimal, preferably one action. Additionally, user interface 8 preferably comprises visual, auditory and/or tactile accessories. User interface 8 may be manufactured in several versions, depending on the nationality and age of the user. For example, different versions of user interface 8 may be labeled in different languages. For children, user interface 8 preferably displays simple instructions which may be transmitted interactively via a communications system.

Besides allowing activation of system 10, user interface 8 can also provide the user with instructions or information. The information is preferably in a form of sensible signals (visual signals, audio signals, vibrations, etc.) and it may include presence, level or absence of harmful agent, operative status and/or impermeability level of system 10.

For example, user interface 8 can generate a first sensible signal when power source 220 is operational, a second sensible signal when power source 220 is not operational, a fourth sensible signal when the components of system 10 are sealed, a fifth sensible signal when the sealing of one or more components of system 10 is broken, a sixth sensible signal for a hardware failure, a seventh sensible signal for a software failure and the like. Optionally and preferably, user interface 8 is powered by an additional power source, hence being operative when power source 220 is not operative or during replacement of power source 220.

According to a preferred embodiment of the present invention the level of the sensible signal is selected to allow sensation of the signal at large distances. This can be done, for example, by integrating a powerful light emitting diode in user interface 8. Powerful light emitting diodes are commercially available and can produce light beam to a distance of over 4 km. Alternatively, user interface 8 may comprise powerful audio units, such as commercially available personal defense audio units, also known as “rape alarms.”

The detection information provided by user interface 8 can be in a form of a color code. For example, a first color (say, red) can indicates that the detected agent is hazardous in a certainty level which is above a predetermined threshold (e.g., about 50%), a second color (say, yellow) can indicates that the detected agent is hazardous in a certainty level which is below the threshold, and a third color (say, green) can indicates that the detected agent is not hazardous.

According to a preferred embodiment of the present invention system 10 may comprise a supplementary sensing unit 356 which preferably continuously monitor environmental conditions. This embodiment is particularly useful when system 10 operates under a triggering or cuing mode. Hence, when the environmental conditions monitored by unit 356 meet a predetermined set of criteria (e.g., a preliminary detection of a potentially harmful agent), unit 356 generates a signal to activation unit 19 to activate system 10. The advantage of this embodiment is that triggering or cuing mode improves the reliability and detection performance of system 10. System 10 may comprise more than one supplementary sensing unit for monitoring different environment conditions.

Hence, in one embodiment, one supplementary sensing unit includes a continuous air sampler coupled to a detector (e.g., a visible light laser) which monitors changes in airborne particle characteristics (such as number and size). Upon realization that such changes occur, additional confirmation may be obtained by additional sensing unit (for example, a laser source) that may provide presumptive information as to the possible source (biological or mineral) of the particulate matter. If the combined information from the two sensing unit correlates to a potentially harmful agent, a full operation of system 10 (e.g., via activation of sensor 18) is initiated.

In another embodiment, supplementary sensing unit 356 comprises a vital signs measuring unit, for measuring vital signs (heart rate, blood pressure, breathing rate, etc.) of the carrier of system 10 as further detailed hereinabove. When the vital signs of meet a predetermined set of criteria (e.g., are reduced below a predetermined threshold) a signal is transmitted to activation unit 19 to activate system 10.

System 10 preferably further comprises an atmospheric condition measuring unit 358 for measuring atmospheric conditions, such as, but not limited to, temperature, barometric pressure, solar radiation, wind speed and direction and relative humidity. This information can be used to predict a propagation path of the detected agent.

According to a preferred embodiment of the present invention communication unit 13 is also used for transmitting information other the aforementioned detection information. Specifically, communication unit 13 can transmit signals representing vital signs, atmospheric conditions, location and the like. Additionally, communication unit 13 can transmit an identification code of system 10, so as to allow the receiving party to identify the source of the signals. The combined transmitted information can be used by the receiving to perform analyses, for example, cross checking the detection information of several detectors and/or identifying clustering.

Communication unit 13 preferably serves also for receiving signals from a central location or from other systems in the communication network. According to a preferred embodiment of the present invention signals, received by communication unit 13, control activation unit 19 to remotely activate or select the operational mode of system 10.

Additionally, the central location can transmit signals to system 10 so as to unilaterally communicate with the user, for example, via a user display 346. Bilateral communication is also contemplated by allowing transmission of response signals from communication unit 13 to the central location. To further facilitate information exchange between system 10 and the communication network, system 10 preferably comprises an image capturing unit 350 (e.g., a stills or video camera) and an input-output audio unit 352 (e.g., a loudspeaker and a microphone).

According to a preferred embodiment of the present invention communication unit 13 is supplemented with at least one communication protocol, tangibly embodied in a readable memory. The communication protocol is preferably configured to allow a takeover of the communication network. This is particularly useful when a harmful agent is detected in a facility which is equipped with an internal communication network, for example, sky-scrapers, shopping malls, governmental building and the like. Upon a detection event in such a facility, the central location can selectively transmit a permission signal to one or more detectors which are carried by key security personnel, which permission signal instructs activation unit 19 to select a takeover mode of system 10. Once the takeover mode is selected each individual of the security personnel can transmit appropriate messages or instructions to the population via the internal communication network. The takeover mode can also be selected manually, or automatically in case of a detection event, as desired.

System 10 is preferably encapsulated in an armored encapsulation 7, which is at least partially impermeable and capable of withstanding extreme thermal and/or mechanical conditions. Specifically, encapsulation 7 protects system 10 from damage due to low temperatures, high temperatures, high humidity, large dust load, extensive rocking and the like. For example, in situations in which system 10 is deployed on water, encapsulation 7 preferably provides system 10 with buoyancy and ensures a safe landing without damage.

According to a preferred embodiment of the present invention system 10 further comprises one or more additional analysis devices 359, such as lab-on-chip devices and the like. Device 359 can be integrated in device 11 or provided as separate units of system 10.

The additional analyses preferably designed to recognize and identify specific biological cells or viruses or specific cell components, e.g., nucleic acids, proteins, membrane components, cell wall components, sugars, lipids and the like. Device 359 can also be designed to detect and analyze characteristic activities (such as enzymatic activities) of an organism or of its components (proteins, including enzymes). Additionally, device 359 can be used to detect and analyze shapes and or motion characteristics of the organisms. Device 359 may comprise one or more reservoir compartments 357 for holding ingredients and reagents for the chemical and biological reactions which may be carried during the analysis. Compartments 357 my contain buffers, tagged- and non-tagged nucleotides, DNA primers, enzymes, antibodies and the like.

Representative example of device(s) 359, include, without limitation, a cell lysis and/or DNA extraction device, a nucleic acid amplification device (e.g., a “PCR on chip” device); a reaction chamber for contacting between collected intact biological cells or viruses and recognition molecules such as antibodies or oligo/polynucleotides; a reaction chamber or surface for contacting between extracts of collected samples, including cell extracts (lysed bacteria, fungi, protozoa, including proteins, enzymes, membrane components, cell wall components, lipids, etc.) and recognition elements or molecules such as antibodies, “molecular imprints or enzyme substrates; a reaction chamber or surface, for contacting between PCR products obtained from DNA extracted from the sample and oligonucleotides of specific or random sequences, singly or in an array format (“DNA chip” or a “DNA array”); a reaction chamber or surface for contacting between DNA extracted from the sample and oligonucleotides of specific or random sequences, singly or in an array format.

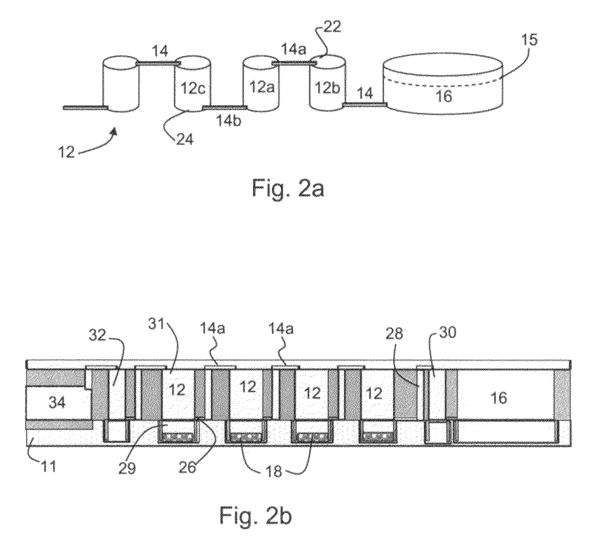

Reference is now made to FIGS. 2a-b which are schematic illustrations of reaction chambers 12 and fluid channels 14, according to a preferred embodiment of the present invention. In an embodiment in which chambers 12 and channels 14 are arranged in one or more sequential arrays.

It is to be understood that the configurations shown in FIGS. 2a-b are not to be considered as limiting and that other arrangements of chambers 12 and channels 14, such as, but not limited to, arrangement facilitating equal filling, are not excluded from the scope of the present invention.

Hence, each reaction chamber in an array (except the first and the last) is preferably in direct fluid communication with at least two other reaction chambers. For example, referring to FIG. 2a, chamber 12a is in direct fluid communication with chambers 12b and 12c, such that chamber 12a is fed from chamber 12b and drained through chamber 12c. In other words, chamber 12b serves as a fluid source for chamber 12a and chamber 12c serves as a fluid sink for chamber 12a. One of ordinary skill in the art would appreciate that with such configuration, and an appropriate transport mechanism, all the reaction chambers in the array are equally filled. For an array of equal-volume reaction chambers, this embodiment ensures equal reaction conditions in all the chambers in the array. In the configuration shown in FIG. 2a, chamber 12a is connected via channel 14a to a top surface 22 of chamber 12b and via channel 14b to a bottom surface 24 of chamber 12c. However, this need not necessarily be the case, since, for some applications, it may be desired to feed and drain all chambers at the same height-level.

FIG. 2b is a schematic illustration of an array of reaction chambers in a preferred embodiment in which chambers 12 are drained through channel 14a (connected at top surface 22) and fed through a channel 14c connected at position 26 which is located above a bottom surface 24 of chambers 12, between a lower part 29 and an upper part 31 thereof. Channels 14a and 14c are in fluid communication preferably via an additional fluid channel, designated 28 in FIG. 2b. Channel 28 can be vertical or can have any orientation with respect to channels 14a and 14c provided the fluid communication therebetween is preserved. As shown in FIG. 2b, in the presently preferred embodiment of the invention, sensors 18 are positioned at bottom surface 24, below positioned 26 so that fluid flow is not substantially obstructed. System 10 may further comprise an input buffer 30, an output buffer 32 and/or an actuator port 34. Input buffer 30 is preferably in fluid communication with sample port 16 and serves as a fluid source for the first reaction chamber in the array. Output buffer 32 is preferably in fluid communication with actuator port 34 and serves as fluid sink for the last reaction chamber in the array. Actuator port 34 can be used to facilitate fluid transport as further detailed hereinbelow.

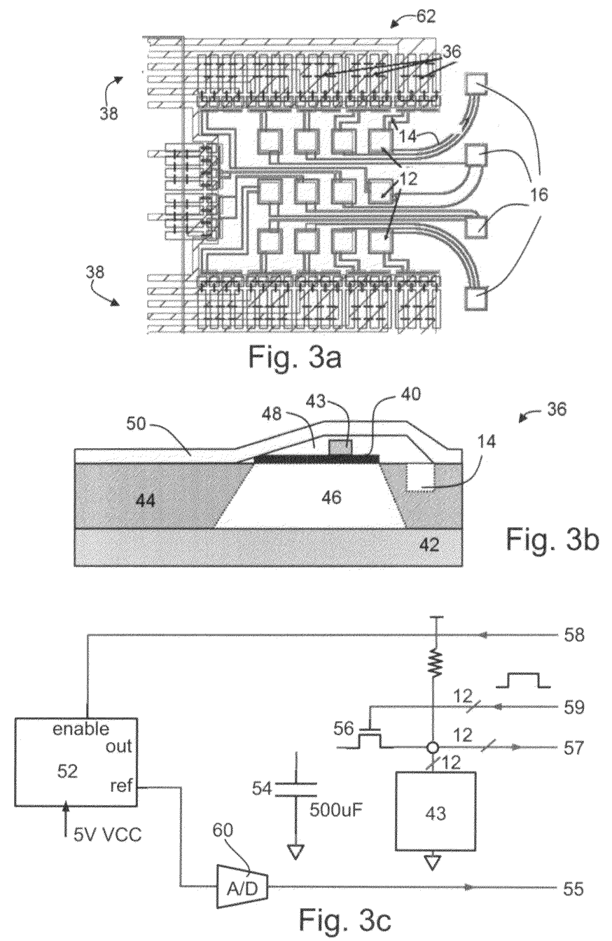

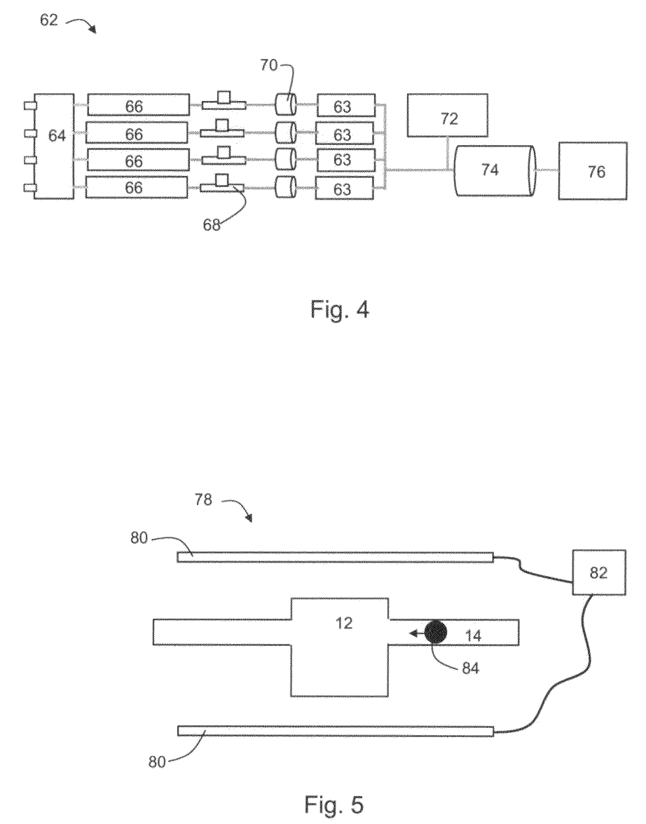

There are many techniques for actuating fluid transport in microchannels. Generally, a transport mechanism 62 is employed (see FIGS. 3a and 4, below). For example, in one embodiment transport mechanism 62 is capable of pumping or injecting sample fluid 15 through channels 14.

Several examples of micro-pumps or micro-injectors which can be utilized in mechanism 62 are known in the art. Mechanism 62 preferably enables sample 15 delivery by applying a under-pressure to actuator port 34, channels 14 or reaction chambers 12, thereby delivering sample 15 from sample port 16 to reaction chambers 12.

As used herein “under-pressure” refers to a pressure value, which is smaller than a pressure value in a reference volume. For example, with respect to sample port 16, “under-pressure” refers to a pressure value which is smaller than the pressure value in sample port 16.

Reference is now made to FIG. 3a which is a schematic illustration of device 11 in the embodiment in transport mechanism 62 comprises a plurality of micro-pumps 36 which are capable of generating a under-pressure in chambers 12 with respect to the pressure in sample port 16 (i.e. the pressure in chambers 12 is lower than the pressure in sample port 16). According to a preferred embodiment of the present invention micro-pumps 36 are controlled by an electric circuitry (not shown, see FIG. 3c), through a plurality of electrical contact, designated 38 in FIG. 3a.

FIG. 3b is a schematic illustration of one of micro-pumps 36. Micro-pump 36 preferably comprises a substrate 42 (e.g., glass substrate) onto which a layer 44 having a vacuum chamber 38 therein is applied. A puncturable membrane 40, is deposited on vacuum chamber 46 thus buffering between vacuum chamber and an additional chamber 48 being in fluid communication with channel 14. Membrane 40 can be made form any suitable materials, such as, but not limited to, silicon-nitride. Additional chamber is sealed by a cover 50, which is preferably, but not obligatory transparent.

When membrane 40 is punctured, the pressure in channels 14 drops thereby actuating flow of sample fluid from sample port 16 to reaction chamber 12. The puncturing of membrane 40 is preferably by a heat shock which can be applied, for example, using a heater 43, controlled by the electronic circuitry (not shown see FIG. 3c) and positioned on or close to membrane 40.

Reference is now made to FIG. 3c, which is a schematic diagram exemplifying a preferred configuration of electronic circuitry 50 controlling micro-pump 36. It is to be understood that the electric configuration of FIG. 3c, as well as the accompanying description is not to be considered as limiting.

Hence, circuitry 50 can have Circuitry 50 includes a DC to DC switching converter 52 which is capable of charging a capacitor 54, having a capacitance of, for example, 500 μF, to a predetermined voltage of, e.g., 18 volts. A control line 58 may be connected to switching converter 52 for enabling or controlling the charging of capacitor 54. Heater 43 can be connected to capacitor 54 through a Metal Oxide Semiconductor (MOS) transistor 56. When a short regulated pulse is applied through line 59 to one of the gates transistor 56, the gate opens and capacitor 54 is discharged through heater 43 thereby initiating the heat shock which punctures membrane 40 as further detailed hereinabove. A typical resistance of heater 43 is about 2Ω, a typical activating current is about 9 A, and a typical a pulse duration is about 20 μs. Circuitry 50 may further comprise several feedback lines. One feedback line, designated 55 can be connected, e.g., via an analog to digital converter 60, to switching converter 52 and can be used for monitoring the status of capacitor 54, another feedback line, designated 57 can be connected to transistor 56 for acquiring an activation status of heater 43, hence to indirectly monitor whether or not membrane 40 is punctured.

FIG. 4 is a schematic illustration of transport mechanism 62 in the embodiment in which external pumping is utilized. For illustrative purposes only, FIG. 4 shows four pumping channels. It is to be understood that any number of pumping channels can be used.

According to a preferred embodiment of the present invention mechanism 62 comprises a pump interface 64 adapted to be connected to actuator port 34 or channels 14 and a vacuum chamber 74, interposed between pump interface 64 and a pumping device 76, and being in fluid communication therewith. In operational mode, pumping device 76 reduces the pressure in vacuum chamber 74, such that vacuum chamber 74 maintains a under-pressure environment. As a result, the pressure in interface 64 and actuator port 34 drops and actuates the flow of sample fluid 15 to reaction chamber 12.

Mechanism 62 may further comprise a pressure sensor 72 for monitoring the pressure at the inlet of vacuum chamber 74. Optionally and preferably, each pumping channel of mechanism 62 may further comprise a valve 63 capable of activating and deactivating the transport of sample fluid. Valve 63 is preferably a fast switching valve. A typical time delay of valve 63 is about 5 ms. The flow parameters (e.g., speed, volume) of sample fluid 15 in each pumping channel is preferably monitored using a flow sensor 66 and regulated using a tap 68. When sample fluid 15 is liquefied, the liquid may case damage to valve 63. According to a preferred embodiment of the present invention, mechanism 62 preferably comprises a filter 70 made of a hydrophobic material which prevents sample fluid 15 from arriving to valve 63. In the hydrophobic material of filter 70, cohesive forces between like molecules dominate over external forces existing between the molecules of the liquid and molecules of filter 70. The free surface of the liquid becomes film-like and the liquid is incapable of wetting filter 70 or penetrating therethrough.

The transport of sample fluid 15 may also be generated by electrical forces. When an uncharged particle (which may be, for example, a drop of sample fluid 15) is placed in a non-uniform electric field, it becomes polarized, i.e., acquires a non-zero electric dipole moment. The interaction between the electric dipole moment and the electric field results in net force acting on the fluid drop, which force is proportional to the electric dipole moment and the gradient of the electric field, and is commonly termed a dielectrophoretic force.

Reference is now made to FIG. 5, which is a schematic illustration of a portion of system 10 in which mechanism 62 comprises an electric field generator 78, according to a preferred embodiment of the present invention. Electric field generator 78 can be any device capable of generating a non-uniform electric field which induces polarization on molecules of sample fluid 15. A representative example include, without limitation, two plates 80 of variable conductivity connected to a voltage source 82. When a voltage is applied to plates 80, dielectrophoretic forces generated by the non-uniform electric field maneuver drop 84 of sample fluid 15 through the fluid channel.

The electric field is preferably designed and configured such that the dielectrophoretic forces direct drop 84 into reaction chambers 12. Alternatively, the electric field is can be designed and configured such that the dielectrophoretic forces direct drop 84 away from reaction chambers 12, for example, when it is desired to maneuver drop 84 from one chamber (e.g., a filled or partially filled chamber) to an empty chamber (e.g., an empty chamber). Still alternatively, the electric field is can be designed and configured such that the dielectrophoretic forces direct one portion of sample fluid 15 into reaction chambers 12 and another portion away from reaction chambers 12, all depending on the desired filling of system 10.

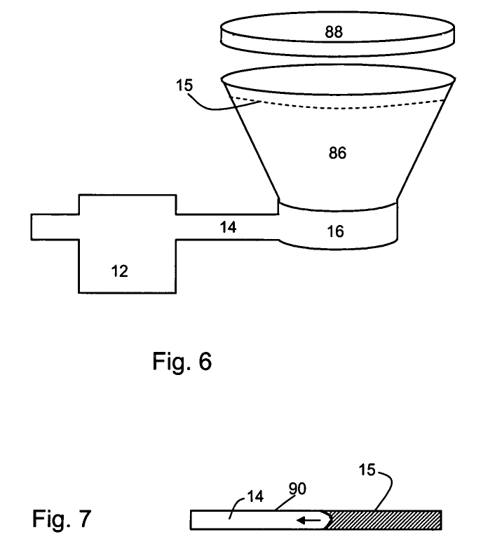

Reference is now made to FIG. 6 which is a schematic illustration of a portion of system 10 in an embodiment in which the transport of sample fluid 15 is generated by hydrostatic pressure. Hence, in this embodiment mechanism 62 preferably comprises a column 86 of sample fluid 15 connected to sample port 16. According to a preferred embodiment of the present invention the height of the column is selected such that the resulting hydrostatic pressure is sufficient for injecting sample fluid 15 into channels 14 and chambers 12. Optionally and preferably, column 86 may be supplemented by a pressing device 88 (e.g., a piston) for further increasing the pressure thereby to improve the flow of sample fluid 15 in channels 14.

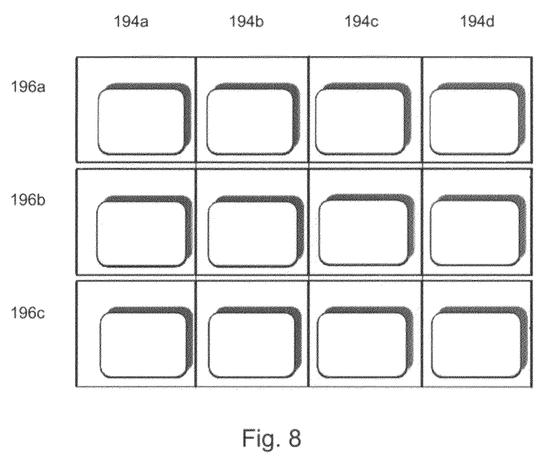

An additional transport technique which is contemplated is transport via capillary action. A capillary action is a phenomenon in which adhesion forces between molecules of the fluid and molecules of solid cause the fluid to flow through a small diameter channel. Hence, referring to FIG. 7, according to a preferred embodiment of the present invention sample fluid 15 flows from sample port 16 into channels 14 via capillary forces generated between sample fluid 15 and walls 90 of channels 14.

In use, a portion or all of the reaction chambers of the sensing device are filled with the sample fluid. The filling can be done by any of the aforementioned transport techniques, e.g., pumping, dielectrophoretic forces, capillary forces, injection and the like. According to a preferred embodiment of the present invention different portions of the reaction chambers of the sensing device can be contain different sensors. In addition, several reaction chambers may not contain sensors at all, thereby serving as a control group.

Similarly, different portions of the reaction chambers can be filled with different fluids, in any combination with the different sensors, so as to allow each sensor to be exposed to a plurality of fluids and each fluid to be sensed by a plurality of sensors. When the different sensors generate different types of signals, several types of detectors (e.g., two or more) are employed, such that each detector is allocated to one chamber, or a group of chambers. The use of several types of detectors is also contemplated for the case in which the same type of signal (e.g., optical signal) is emitted from all chambers,

According to a preferred embodiment of the present invention a continues flow of the sample fluid in the channels is maintained, e.g., using a circulation mechanism 192, so as continuously replace the sample fluid the reaction chambers. Continues flow of sample fluid is advantageous for online monitoring and detection of the sample fluid. Thus, according to a preferred embodiment of the present invention the detection of the generated signals is done substantially in real time.

In an online measurement, it is often desired to have an indication of the general state of the sensing device. The electronic circuitries of the device (e.g., the aforementioned circuitries for controlling the transport mechanism, temperature, light source, detector, etc.) can be monitored substantially in real time by incorporating appropriate feedback lines therein. In addition, several sensors preferably generate a reference signal at all times, so as to provide indication of their operation.

When wetware is used in the sensing device, the viability thereof can be monitored by incorporating in a few reaction chambers, a material (e.g., a biological material) which generate a detectable reference signal at all times. The reference signal can be optical, electrical electrochemical or any other signal.

The present embodiment has several advantages. First, the reference signal indicates viability of the sensing device. For example, lack of reference signal can indicate that the wetware is significantly damaged.

Second, when the sample is highly toxic, the wetware may fail to produce the detectable signal, for example, when a highly toxic substance is present in the chambers. On the other hand, highly toxic sample may also prevent the wetware from generating the reference signal. Thus, in this case a cessation of the reference signal indicates a highly toxic sample.

Third, the reference signal can serve as a sensor for abnormal state of the sample. For example, when the reference signal is generated by live cells, an abnormal sample state (e.g., abnormal temperature, abnormal pH level, etc.) may cause a stress to the live cells resulting in a decrease of their ability to generate normal reference signal. A reference signal which is below a predetermined level therefore indicates an abnormal sample state.

Thus, the combination of the reference signal and the sensors of the device allows an efficient detection of the agent in the sample. According to a preferred embodiment of the present invention, the presence, concentration and/or type of the agent can be determined. More specifically, the presence of agents in the sample can be determined by detecting a change in signal reading or a change the rate of change of signal readings (both signal in response to the agent and reference signals); the concentration of the agent in the sample is determined by the absolute value of the detected signal; and the type of agent is determined from the imaging information provided by the detector.

As stated, different portions of the reaction chambers of the sensing device can be contain different sensors and/or be filled with different fluids, in any combination with the different sensors. For example, in the embodiment in which the reaction chambers and the fluid channels are arranged in sequential arrays (see FIGS. 2a-b), each array can be allocated for a different sample fluid, while each reaction chamber in a particular array contain a different sensor. Thus, when a particular sample fluid is transported into the chambers of its respective array, different signals are generated in different reaction chambers, thus enable multiplexing.

Reference is now made to FIG. 8, which is a schematic illustration of a logical and physical division of the sensing device, according to the presently preferred embodiment of the invention. As show in FIG. 8, the sensing device can be divided into rows and columns. FIG. 8 exemplify four columns, designated 194a, 194b, 194c and 194d, and three rows, designated 196a, 196b and 196c. The reaction chambers of each column can be in fluid communication thereamongst, so as to allow filling at the reaction chambers of a particular column with the same sample fluid.

The columns can be physically divided so as to prevent sample fluids from flowing across a row of reaction chambers. Each row can be allocated to a different functionality. For example, row 196a can be allocated for signal stability testing, row 196b can be allocated for detecting unknown agents and row 198c can be allocated for detecting known agents.

Different columns can be allocated for different sample fluid or the same sample fluid as desired. For example, prior to the transport of the sample fluid into one column, the sample fluid can be purified, e.g., using an activated filter, thus to serve as a control to another column in which no purification was employed.

Following is a description of the principles and operations of detector 108 in the embodiment in which detector 108 is a planar light detector. Hence, referring again to FIGS. 1a-b, detector 108 receives an optical signal 106 from sensors 18 and converts signal 106 into electronic signals (e.g., analog or preferably digital) which in turn can be received and analyzed, for example, by data processor 23. Detector 108 preferably detects optical signals 106 simultaneously from several reaction chambers. More preferably, detector 106 detects optical signals 106 simultaneously from all the reaction chambers.

Data processor 23 is preferably designed to include software for determining the presence, absence or concentration of the agent in sample fluid 15. For example, data processor 23 can determine whether or not sample fluid 15 is toxic and send an appropriate signal to communication unit 13 and/or the user which can monitor the signal e.g., using display 346. Data processor 23 can also calculate the concentration of the agent in sample fluid 15 and provide the information to communication unit 13, display 346 or any other suitable component in system 10.

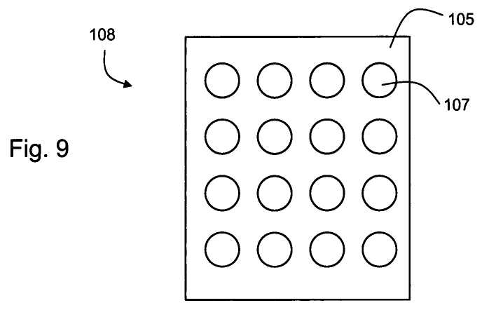

Reference is now made to FIG. 9, which is a schematic illustration of detector 108, according to a preferred embodiment of the present invention. Detector 108 preferably comprises a matrix 105 having a plurality of addressable elementary units 107, each being capable of converting light into electrical signal. Each elementary unit is allocated for a specific reaction chamber. When optical signal 106 originating from a particular reaction chamber impinges on matrix 105, the respective elementary unit generates a signal, which can then be analyzed by data processor 23. The signal generated by elementary units 107 preferably includes imagery information so as to allow attributing each signal to a respective reaction chamber, thereby providing an image thereof. Thus, according to a preferred embodiment of the present invention detector 108 is capable of providing an image of the sensors which generate optical signals 106.

Several types of elementary detection units are contemplated herein. For example, elementary units 107 can be positive-intrinsic-negative (PIN) photodiodes. A PIN photodiode is a device having a large, neutrally doped intrinsic region sandwiched between p-doped and n-doped semiconducting regions. A PIN diode exhibits an increase in electrical conductivity as a function of the intensity, wavelength and modulation rate of incident radiation. The avalanche photodiode, is preferably used in accordance with the present invention since it is capable of generating an amplified current by avalanche multiplication in which electrons, initially generated by the incident light, accelerate and collide with other electrons.

Detector 108, which incorporates PIN photodiodes or avalanche photodiodes enables accurate monitoring of intensity as well as the wavelength of optical signal 106.

According to an alternative embodiment, detector 108 employs a charge-coupled device (CCD), in which elementary units 107 are silicon chips. When light hits the silicon chip, electrons are released from the crystalline structure of the silicon and deposited into small units or wells. Once the image is captured, the electrons in the wells are sent into a recorder where they are counted.

In another embodiment, detector 108 comprises at least one photomultiplier. Typically, a photomultiplier is a vacuum tube including a photocathode which is capable of converting light into electrons, by virtue of the photoelectric effect, an electron multiplier and an anode. When light enters the photocathode, the photocathode electrons are emitted into the vacuum and then directed by a system of focusing electrode towards the electron multiplier. The electron multiplier is a string of successive electron absorbers with enhanced secondary emission hence multiply the numbers of electrons. The amplification of the electron multiplier can reach eight orders of magnitude. Once multiplied, the electrons are collected by the anode as an output signal. Because of the high secondary-emission multiplication, the photomultiplier provides extremely high sensitivity and low noise.

According to yet another alternative embodiment, detector 108 employs complementary metal oxide semiconductor (CMOS) technology. The advantage of using the CMOS technology is that the elementary units and various quantification parts can be integrated into a single device, which may be compact and simple to operate. Such CMOS are commercially available such as for example the ACS-1394 fire-wire camera based on the ACS-1024 CMOS Image Sensor manufactured by Photonics Vision Systems, or IBIS4 CMOS Image Sensor manufactured by Fill Factory http://www.fillfactory.com). Further description of a CMOS imaging sensor which can be used as detector 108, is provided in the Examples section which follows.

Referring again to FIG. 1b, according to a preferred embodiment of the present invention system 10 further comprises at least one temperature control unit 25, for controlling the temperature of system 10. For example, temperature control unit 25, can monitor and adjust the temperature of device 11 and/or detector 108 so as to optimize their operation. Temperature control unit 25 can be, for example, a thermoelectric device.

A thermoelectric device is a device that either converts heat directly into electricity or transform electrical energy into pumped thermal power for heating or cooling. Such a device is based on thermoelectric effects involving relations between the flow of heat and electricity through solid bodies. Generally, a thermoelectric device comprises at least one pair of dissimilar metals. When the device is used for cooling or heating, a potential difference is applied on the dissimilar metals and heat is pumped from one metal to the other. When the device is used for converting heat to electricity (e.g., for the purpose of monitoring the temperature of an object relative to a reference environment), the two metals are kept at different temperatures, and a potential difference is produced across.

Other temperature control units include, but are not limited to, liquid coolers, gas coolers, blowers and the like.

When sensors 18 include fluorescent material, optical signal 106 is generated in response to an excitation light 100, emitted by a light source 120. Several configurations for positioning light source 120 are contemplated, depending on the relative angle between the detected portion of optical signal 106 and excitation light 100. For example, in one embodiment light source 120 is positioned on the side of detector 11, such that the detected portion of optical signal 106 is substantially perpendicular to excitation light 100. In another embodiment light source 120 is positioned above or below device 11 in a manner such that device 11 is between light source 120 and light detector 108. In this embodiment, the detected portion of optical signal 106 is substantially parallel to excitation light 100. In an alternative embodiment, light source 120 is positioned between device 11 and light detector 108. In this embodiment, the detected portion of optical signal 106 is substantially anti-parallel to excitation light 100.

It is to be understood that, although FIG. 1b show light detector 108 positioned on the side of device 11, this configuration is not to be considered as limiting.

The low fluorescence quantum yield of presently available fluorescent materials requires a separation between the optical signal and the excitation light. According to a preferred embodiment of the present invention the separation of the excitation light from the optical signal can be spatial separation and/or in the spectral separation.

As used herein, the term “spatial separation” refers to confinement of light energy to propagate only in a predetermined volume, irrespective of its wavelength, and the term “spectral separation” refers to absorption or reflection of certain wavelengths and transmission of other wavelengths.

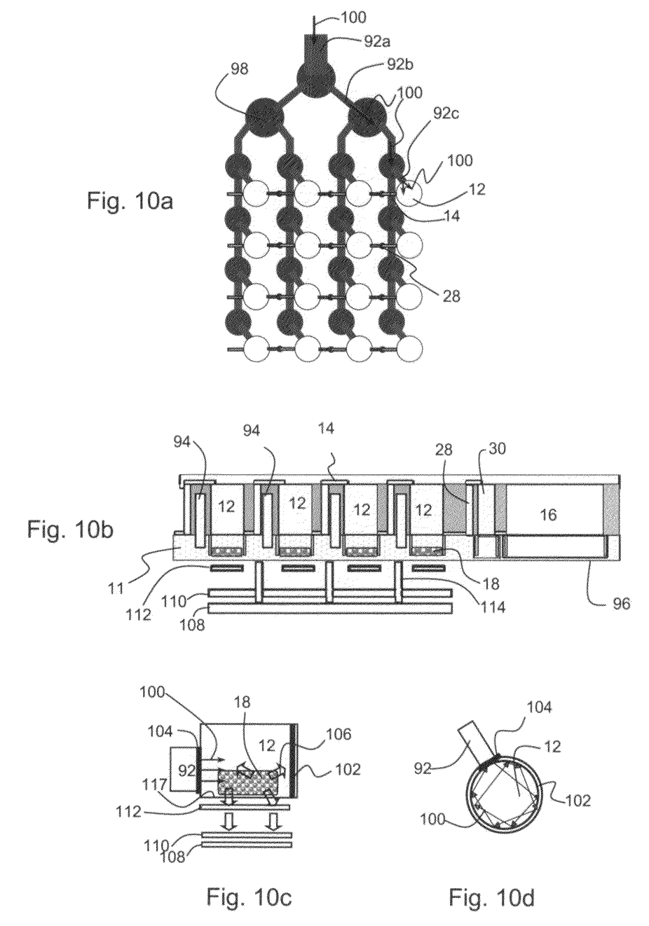

Reference is now made to FIG. 10a-d, which are schematic illustrations of device 11 in an embodiment in which spatial separation of the excitation light from the optical signal is employed. According to the presently preferred embodiment of the invention, device 11 comprises a plurality of waveguides 92 distributing excitation light 100 among chambers 12.

As the present embodiment relies upon the ability to transmit and emit light through a waveguide, a brief description of such technology is provided hereinbelow.

The technology to transmit and guide light rays through optical systems exploits a physical phenomenon known as total internal reflection, in which a light is confined within a material surrounded by other materials with lower refractive index. When a ray of light moves within a transparent substrate and strikes one of its internal surfaces at a certain angle, the ray of light can be either reflected from the surface or refracted out of the surface into the open air in contact with the substrate. The condition according to which the light is reflected or refracted is determined by Snell's law, which is a mathematical relation between the impinging angle, the refracting angle (in case in case of refraction) and the refractive indices of both the substrate and the air. Broadly speaking, depending on the wavelength of the light, for a sufficiently large impinging angle (also known as the critical angle) no refraction can occur and the energy of the light is trapped within the substrate. In other words, the light is reflected from the internal surface as if from a mirror. Under these conditions, total internal reflection is said to take place.

Many optical devices operate according to the total internal reflection phenomenon. One such optical device is the optical fiber. Optical fibers are transparent flexible rods of glass or plastic, basically composed of a core and cladding. The core is the inner part of the fiber, through which light is guided, while the cladding surrounds it completely. The refractive index of the core is higher than that of the cladding, so that light in the core impinging the boundary with the cladding at a critical angle is confined in the core by total internal reflection.

As stated, total internal reflection occurs only for light rays impinging the internal surface of the optical fiber with an angle which is larger than the critical angle. Thus, a calculation performed according to geometrical optics may provide the largest angle which is allowed for total internal reflection to take place. An important parameter of every optical fiber (or any other light transmitting optical device) is known as the “numerical aperture,” which is defined as the sine of the largest incident light ray angle that is successfully transmitted through the optical fiber, multiplied by the index of refraction of the medium from which the light ray enters the optical fiber.

Another optical device designed for guiding light is the graded-index optical fiber, in which the light ray is guided by refraction rather than by total internal reflection. In this optical fiber, the refractive index decreases gradually from the center outwards along the radial direction, and finally drops to the same value as the cladding at the edge of the core. As the refractive index does not change abruptly at the boundary between the core and the cladding, there is no total internal reflection. However, although no total internal reflection takes place, the refraction bends the guided light rays back into the center of the core while the light passes through layers with lower refractive indexes.

Optical fibers are available in various lengths and core-diameters. For large core diameters, glass optical fibers are known to be more brittle and fragile than plastic optical fiber.

Another type of optical device is based on photonic materials, where the light ray is confined within a band gap material surrounding the light ray. In this type of optical device, also known as a photonic material waveguide, the light is confined in the vicinity of low-index region. One example of a photonic material waveguide is a silica fiber having an array of small air holes throughout its length. This configuration is capable of providing lossless light transmitting, e.g., in either cylindrical or planar type waveguides.

Thus, according to a preferred embodiment of the present invention, each of waveguides 92 can be an optical fiber, a graded-index optical fiber a photonic material or any other optical device capable of transmitting light.

It is expected that during the life of this patent many relevant technologies for guiding light will be developed and the scope of the term waveguide is intended to include all such new technologies a priori.

Irrespectively of their type and operation principle, waveguides 92 can be integrated with or formed in body or substrate 17 of device 11. Alternatively, device 11 may be manufactured with a plurality of grooves 94 (see FIG. 10b) sizewise compatible with waveguides 92 such that waveguides 92 are inserted into grooves 94 prior to the excitation procedure. This embodiment is particularly useful when device 11 is made of a disposable material and it is desired to keep waveguides 92 for additional uses once device 11 is replaced.

As shown in FIG. 10a, waveguides 92 are preferably arranged in a multi-furcated arrangement (“a tree”), having a plurality of light splitting junctions 98, such that excitation light 100 enters through a single primary waveguide, designated 92a, and distributed by light splitting junctions 98 to secondary waveguides, designated 92b and 92c. Each light splitting junction 98 is preferably designed to satisfy the numerical apertures of its outgoing waveguides. Waveguides 92 may also be arranged in several multi-furcated trees, so that excitation light 100 can enter system 10 through several primary waveguides. This embodiment is useful when several excitation wavelengths are used, whereby each multi-furcated tree of waveguides is dedicated to a particular excitation wavelength.

In any event, according to a preferred embodiment of the present invention waveguides 92 distribute the light in a manner such that impingement of the excitation light on sensors 18 is maximized and impingement of excitation light 100 on a surface 96 of substrate 17 is minimized. Once the fluorescent material in sensors 18 is excited by light 100, an optical signal 106 is generated and can be detected, for example, using a light detector 108 position in the light path of signal 106, as further detailed hereinafter.

The minimization of impingement on surface 96 and the maximization of impingement on sensors 18 can be better understood from FIGS. 10c-d which illustrate a side view (FIG. 10c) and a top view (FIG. 10d) of one waveguide 92 guiding light 100 into chamber 12.

Referring to FIG. 10c, the minimization of the impingement of excitation light 100 on surface 96 can be achieved by imposing a predetermined propagation direction on light 100. More specifically, according to a preferred embodiment of the present invention, when exiting waveguide 92 and entering chamber 12, light 100 propagates a direction which is substantially parallel to surface 96.

Referring to FIG. 10d, the maximization of impingement of light 100 on sensors 18 can be achieved by allowing light 100 to exit waveguide in a plurality of co-planar direction, each being parallel to surface 96. A predetermined propagation direction can be imposed on light 100 either directly by waveguide 92 or by one or more additional optical elements 104, e.g., a diffraction grating, a reflection grating, a mini-prism and the like. Optionally and preferably chamber 12 my comprise a reflective coat 102, covering the walls of chamber 12, so as to reflect light 100 hence to further increase the impingement of light 100 of sensors 18. As further detailed hereinunder and in the Examples section that follows, sensors 18 may be biological sensors. In this embodiments coat 102 is preferably made of a biocompatible material.

Thus, excitation light 100 is constrained to propagate in a predetermined direction while an optical signal 106, generated by sensors 18 in response to light 100, is allowed to propagate in all directions. According to a preferred embodiment of the present invention at least one side of chamber 12 (e.g., the bottom side 117) is not coated by coat 102. One of ordinary skill in the art would appreciate that with such configuration, optical signal 106 can be detected by detector 108 without being screened by excitation light 100 which is substantially confined in chamber 12.

It is appreciated, however, that light 100 can be diverted, for example, when light 100 is not absorbed by sensors 18 but rather being scattered to a different direction. Thus, according to a preferred embodiment of the present invention, the aforementioned spatial separation is combined with spectral separation. For example, an emission filter 110 can be positioned in the light path of optical signal 106 so as to prevent diverted rays of excitation light 100 from arriving to detector 108. Emission filter 110 preferably allows transmission of optical signal 106 substantially without loses. Additionally or alternatively, coat 102 can be a selective coat, capable of selectively reflecting light of a particular wavelength. In this embodiment, coat 102 may cover also bottom side 107 of chamber 12 so that optical signal 106 is transmitted therethrough and light 100 is reflected thereby.

According to a preferred embodiment of the present invention, optical signals generated in different reaction chambers are spatially separated so as to prevent cross talks between the different optical signals. This can be done, for example, by positioning an optical focusing device 112 (e.g., a microlens) in the light path of optical signal 106 so as to focus signal 106 on detector 108. Alternatively device 112 can be positioned so as to collimate signal 106 to a predetermined direction. In this embodiment, the optical signals of different chambers are preferably collimated to propagate in parallel directions thereby preventing cross talks therebetween. Optionally, a plurality of optical separations 114 can be positioned between different optical signals so as confined each optical signal not to cross the light path of the other optical signals. Optical separation 114 can be, for example, a reflector to minimize losses.

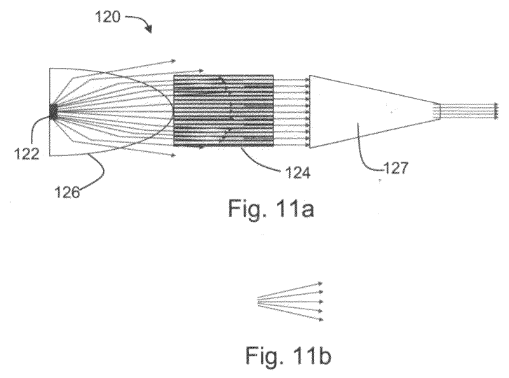

Reference is now made to FIG. 11a, which is a schematic illustration of a side view of light source 120, in the embodiment in which waveguides 92 are employed. According to a preferred embodiment of the present invention light source 120 comprises a light emitting device 122, a collimator 124 and a light coupler 127. Light emitting device 122 can be, for example, a light emitting diode (LED), covered by a collimating lens 126, capable of partially collimating the light emitted by the LED. Typically, lens 126 has a diameter of about 5 millimeters and is capable of providing a beam having a divergence of about 15°. Collimator 124 serves for further collimating light 100 and light coupler 127 serves for reducing the diameter of the light beam so as to facilitate coupling of light 100 into waveguide 92. Light coupler 127 can be any known device for coupling a light into a waveguide in a manner that the impinging angle of the light on the waveguide satisfies its numerical aperture. One such light coupler which is commercially available is known as a “pigtail.”

FIG. 11b, schematically illustrates a top view the light beam outputted by light source 120. As shown in FIG. 11b, the light beam propagate in a plurality of co-planar direction thus maximizing the impingement of light 100 on sensors 18 as further detailed hereinabove.

Typically but not obligatory, light emitting device 122 emits blue light (e.g., wavelength of about 470 nm) at an optical power of about 5 mW. Typical dimensions of light emitting device 122 are about 5 mm in width and about 10 mm in length.

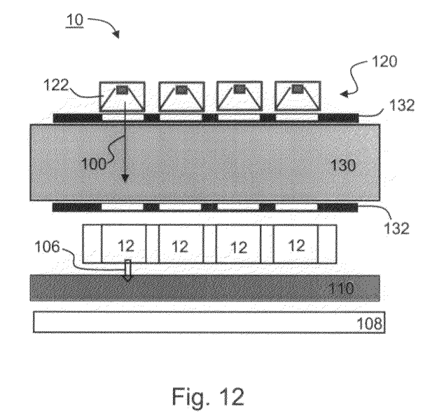

Reference is now made to FIG. 12 which is a schematic illustration of a portion of system 10 in an embodiment in which device 11 is positioned between light source 120 and detector 108. Thus, in this embodiment, the detected portion of optical signal 106 is substantially parallel to excitation light 100.

It is recognized that when optical signal 106 is parallel to excitation light 100, a spectral separation between optical signal 106 and excitation light 100 is required. Thus, system 10 preferably comprises one or more selective filters for selectively allowing transmission of light having predetermined wavelength. One such selective filter is preferably an excitation filter 130 which allows transmission of excitation light 100 and substantially prevents transmission of light having different wavelengths. Another such selective filter is the aforementioned emission filter 110 which allows transmission of optical signal 106 and substantially prevents transmission of light having different wavelengths.

To facilitate substantially simultaneous excitation of the sensors in all or at least a portion of chambers 12, light source 120 preferably comprises a plurality of light emitting devices, which can be, for example, similar to the aforementioned light emitting device 122. Optionally and preferably, system 10 comprises one or more separators 132 for substantially preventing cross talks between different excitation light rays.

To provide a better separation between excitation light rays emitted by different light emitting devices, system 10 can employ an arrangement of optical fibers, as further detailed hereinbelow.

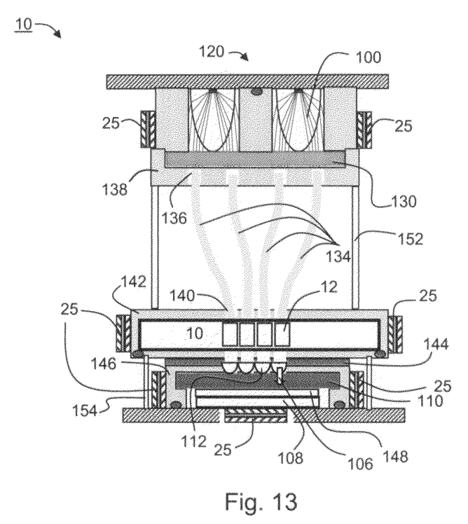

Reference is now made to FIG. 13, which is a schematic illustration of a portion of system 10 in an embodiment in which a plurality of external optical fibers are employed. Generally, in this embodiment system 10 comprises a first housing 138, holding light emitting devices 122 and excitation filter 130, a second housing 142, holding device 11, and a third housing 146, holding detector 108 and emission filter 110. First hosing 138 is preferably connected to second housing 142 by supporting legs 152, and second housing 142 is preferably connected to third housing 146 by supporting legs 154. Alternatively, housings 138, 142 and 146 can be fixed to encapsulation 62 (not shown, see FIG. 1a).

According to the presently preferred embodiment of the invention, system 10 comprises a plurality of optical fibers 134 which deliver excitation light 100 from emitting devices 122 to sensing device 11. Preferably, as shown in FIG. 13, each optical fiber delivers excitation light 100 to one of chambers 12. Alternatively, one optical fiber can deliver light 100 to more than one chamber. A first end of each of optical fibers 134 is preferably connected, e.g., via a groove 136, to housing 138 and a second end thereof is preferably connected, e.g., via a groove 140, to second housing 142.

First housing 138 and second housing 142 are preferably made of a thermally conductive material, so as to allow temperature control unit 25 to monitor and control the temperatures thereof. Optionally, system 10 can comprise one or more thermistors being in thermal communication with first 138 and/or second 142 housings, for sensing the temperatures.

Second housing 142 is preferably thermally isolated from detector 108. This can be done, for example, using one or more thermal isolators 144 positioned adjacently to second housing 142 and/or third housing 146.

Optionally an additional filter, an infrared filter 148, can be positioned in the light path of optical signal 106 so as to filter out infrared radiation which may be generated by third housing 146, when its temperature is rising.

As stated, optical signals generated in different reaction chambers can be spatially separated, for example, using an optical focusing device 112, so as to prevent cross talks between the different optical signals. Representative examples of device 112 include without limitation, a lens and a plurality of lenses (e.g., a micro-lens matrix). Device 112 can be positioned on second 142 or third housing 146. Similarly to the above description, device 112 can either focus optical signal 106 on detector 108 or collimate optical signal 106 such that optical signals of different chambers propagate substantially in parallel directions.

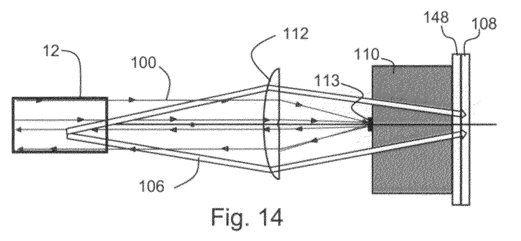

Optical focusing device 112 can also be used for further separation of excitation light 100 from optical signal 106. This can be better understood from FIG. 14, which is a schematic illustration of the light path of excitation light 100 once entering chamber 12. FIG. 14 is rotated anticlockwise by 90° relative to FIG. 13.

Due to the use of optical fibers 134, the rays of light 100 are substantially parallel. When light 100 enters chamber 12 it can (i) absorbed by sensors 18 which in response emits optical signal 106; (ii) scatter off sensors 18 and continue to propagate in a diverted direction; or (iii) continue to propagate in its original direction without interacting sensors 18. Focusing device 112 is preferably oriented in a manner such that the parallel, non-interacting, light rays are focused by focusing device 112 to its focal point. According to a preferred embodiment of the present invention an opaque object or a reflector 113 is positioned in the focal point of focusing device 112 so as to absorb or reflect light 100 hence to prevent it from arriving to detector 108. Reflector 113 is preferably sufficiently small so as not to absorb or reflect off-focal rays. Unlike light 100, optical signals 106 are emitted and propagated in a plurality of directions, so that only a small portion of optical signals 106 is focused to the focal point of focusing device 112. Being sufficiently small, the effect of reflector 113 is negligible for optical signals 106. On the other hand, excitation light rays which are scatter off sensors 18 without being absorbed thereby arrive to focusing device 112 in a direction which may be not parallel to its focal axis. Such non-parallel rays, however, are absorbed by emission filter 110.

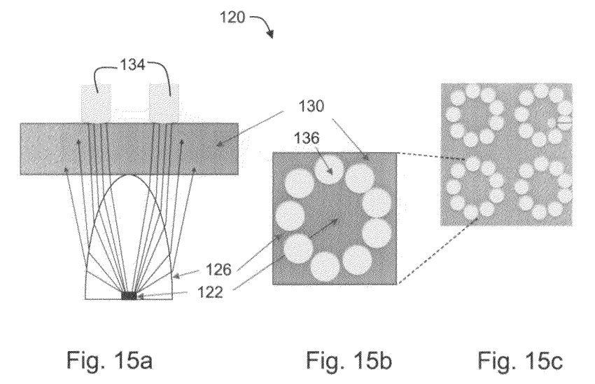

Reference is now made to FIGS. 15a-c, which are schematic illustrations of light source 120 in the embodiment of system 10 in which external optical fibers 134 are employed. Hence, light source 120 preferably comprises a plurality of light emitting devices 122 covered by collimating lens 126 and arranged in a manner that light emitted by each one of devices 122 enters on one or more of optical fibers 134. For example, a plurality of grooves 136 can be circularly arranged in front of a collimating lens of a single light emitting device, such that light rays having a predetermined impinging angle enter the optical fibers. One of ordinary skill in the art will appreciate that the circular arrangement of grooves 136 ensures that each optical fiber is impinged by the light substantially at the same angle. Referring to FIG. 15c, when a plurality of light emitting devices 122 is used, each device can provide excitation light to many optical fibers hence also to many reaction chambers. For example, in the embodiment shown in FIG. 15c, there are four light emitting devices, each providing excitation light to nine optical fibers, hence to nine reaction chambers.

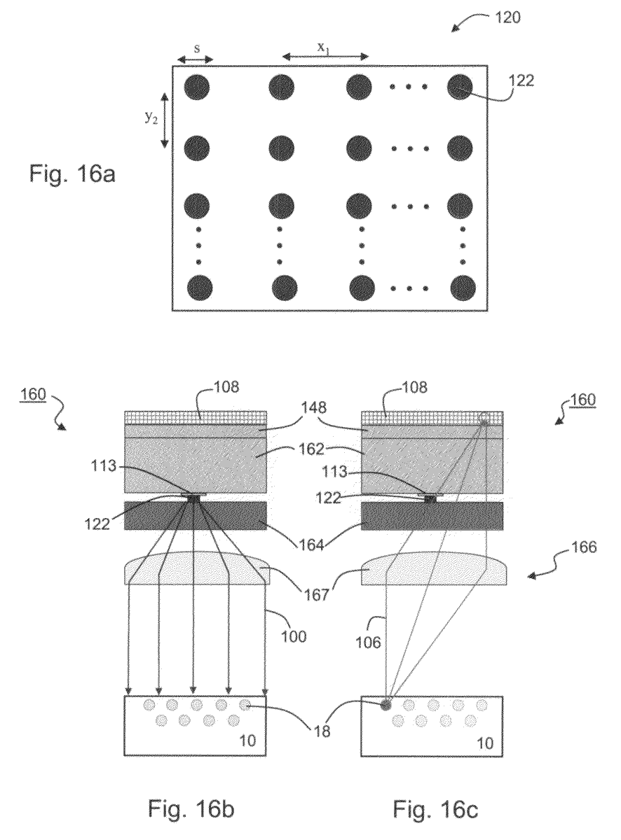

Reference is now made to FIGS. 16a-c which are schematic illustrations of a an optical setup of system 10 in the embodiment in which light source 120 is positioned between device 11 and light detector 108. In this embodiment, system 10 preferably further comprises an apparatus 160 for imaging the pattern of optical signals 106. Apparatus 160 comprises detector 108, an optical element 166 which may be, for example, a plurality of lenses 167 and light source 120. Lenses 167 are preferably arranged in an arrangement which is compatible with the arrangement of chambers 12, such that each lens is allocated to a predetermined number of chambers (e.g., one lens per chamber).

Referring to FIG. 16a, light source 120 can be an arrangement of light emitting devices 122, which is preferably compatible with the arrangement of lenses 167 such that each light emitting device is allocated to a predetermined number of lenses (e.g., one light emitting device per lens). Shown in FIG. 16a is a rectangular arrangement of light emitting devices 122, in which the distance between two adjacent light emitting devices is x₁ in one direction (say, the “x” direction) and y₁ in the orthogonal direction (say, the “y” direction). Typical value for both x₁ and y₁ is a few millimeters, for example, 1 millimeter. x₁ can be equal to, or different from y₁, depending on the desired geometrical arrangement, for example, the density of light emitting devices 122 in the respective direction. The transverse size of each light emitting device, designated s in FIG. 16a, is typically from about 10 μm to about 20 μm. Light emitting devices 122 can be activated simultaneously or independently.

FIGS. 16b-c show one light emitting device and one lens respectively designated by numerals 122 and 167. Detector 108 is preferably connected to a first substrate 162, which may be, for example, a glass substrate coated so as to prevent randomly reflected excitation rays from penetrating therethrough. In embodiments in which infrared filter 148 is employed, infrared filter 148 is preferably formed on first substrate 162 and detector 108 is connected to infrared filter 148. Light emitting device 122 is preferably connected to a second substrate 164, which can be, for example, a sapphire substrate.

According to a preferred embodiment of the present invention light emitting device 122 is configured to generate excitation light 100 in a direction other than a direction of detector 108. This can be done, for example, by positioning opaque object or reflector 113 adjacently to light emitting device 122, between light emitting device 122 and detector 108 thereby to prevent light 100 from impinging on detector 108. Reflector 113 can also have a non planar shape (e.g., parabolic or hyperbolic shape) so as to increase the amount of excitation light propagating in the direction of system 10.

FIG. 16b show the light path of excitation light 100. As shown, light emitting device 122 is preferably positioned at the focal point of lens 167, so that excitation light 100 is collimated by lens 167, and impinges on sensors 18 of system 10 in a form of a collimated beam.

FIG. 16c show the light path of optical signals 106, emitted by sensors 18. According to a preferred embodiment of the present invention, lens 167 is positioned in a manner such that optical signals 106 are focused by lens 167 to impinge on detector 108. This can be achieved by positioning lens 167 half way between detector 108 and system 10, at two focal distances therefrom. Only collimated light rays have a light path which goes through the focal point of lens 167. Thus, being emitted at a plurality of directions, a large portion of optical signals 106 arriving at lens 167 is not collimated, and therefore is not affected by reflector 113.

The above selection of two focal distances between lens 167 and system 10 on the one side, and between lens 167 and detector 108 on the other side, ensures mapping of sensors 18 or chambers 12 on detector 108. More specifically, each one of reaction chamber 12 is represented by an addressable region on detector 108. When sensors of a particular reaction chamber emit optical signal 106 detector 108 detects this signal at the respective addressable region. Thus, an image the emitting sensors is formed on detector 108. Knowing the reactivity properties of the sensors of the respective reaction chamber in device 11, the image can be used, for example, by data processor 23 (not shown, see FIG. 1a), for determining the presence and/or concentration of the agent(s) with which the sensors react.

As stated, the sensors which are employed by the present invention are capable of generating a detectable signal when exposed to the agents in the sample. According to a preferred embodiment of the present invention sensors 18 are biological sensors. Many biological sensors are contemplated. Preferably the biological sensors are made of a biological material (e.g., cell population) capable of producing a material when exposed to the agent. Representative examples of the produced material include, without limitation, a bioluminescent material, a phosphorescent material and a fluorescent material. Alternatively, the biological sensors can produce a material which is capable of altering the electrostatic characteristic of the sample.

Although numerous examples of biological sensors exist in the art, these are limited by instability of the biological component, irreversibility, costs of production and limited ability to identify broad range of agents.

To overcome such limitations, the present Inventors have devised and constructed a reporter-expressing cell population which is composed of discrete subpopulations each capable of expressing the reporter in response to a different agent or groups of agents. When exposed to an agent, the various subpopulations produce a specific expression pattern which forms a signature profile specific to the agent present in the sample. To enable such agent specific expression, the present inventors carefully selected a group of promoters which can be activated by different agents from a number of promoter libraries. It is postulated herein that by utilizing a broad range of physiologically-responsive promoters, one increases an ability of a cell population transformed with reporter constructs containing such promoters to uniquely respond (via unique reporter expression patterns) to each of a broad range of agents.

The present embodiments successfully provide a population of cells which can be utilized as biological sensors. The population of cells is composed of at least two subpopulations of cells. A first such subpopulation includes a first reporter expression construct which is capable of reporter expression when the cells of this subpopulation are exposed to a first agent. A second subpopulation of cells includes a second reporter expression construct which is capable of reporter expression when the cells of the second subpopulation are exposed to a second agent.

As used herein “population of cells” refers to prokaryotic or eukaryotic cells which can be genetically modified (in a transient or stable manner) to express exogenous polynucleotides.

Examples of prokaryotic cells which can be used in accordance with this embodiment include but are not limited to bacterial cells, such as Pseudomonas, Bacillus, Bacteriodes, Vibrio, Yersinia, Clostridium, Mycobacterium, Mycoplasma, Coryynebacterium, Escherichia, Salmonella, Shigella, Rhodococcus, Methanococcus, Micrococcus, Arthrobacter, Listeria, Klebsiella, Aeromonas, Streptomyces and Xanthomonas.

Examples of eukaryotic cells which can be used in accordance with the present embodiment include but are not limited to cell-lines, primary cultures or permanent cell cultures of fungal cells such as Aspergillus niger and Ustilago maydis [Regenfelder, E. et al. (1997) EMBO J. 16:1934-1942], yeast cells (see U.S. Pat. Nos. 5,691,188, 5,482,835), such as Saccharomyces, Pichia, Zygosaccharomyces, Trichoderma, Candida, and Hansenula, plant cells, insect cells, nematoda cells such as c. elegans, invertebrate cells, vetebrate cells and mammalian cells such as fibroblasts, epithelial cells, endothelial cells, lymphoid cells, neuronal cells and the like. Cells are commercially available from the American Type Culture Co. (Rockville, Md.).

As mentioned hereinabove, the population of cells preferably includes at least two subpopulations of cells. However, it is appreciated that the more subpopulations included in the cell population the higher the chances of such a cell population to accurately identify agents present in a sample exposed thereto.

As mentioned hereinabove, each subpopulation of cells includes a reporter expression construct, which expresses a detectable reporter molecule when the cell is exposed to an agent.

As used herein “reporter expression construct” refers to a vector which includes a polynucleotide sequence encoding a reporter. The reporter expression construct is preferably designed to randomly integrate into the genome of the cell, such that expression of the reporter polypeptide is governed by an endogenous regulatory element which is inducible by an agent.

According to a preferred embodiment of the present invention, the polynucleotide sequence is positioned in the construct under the transcriptional control of at least one cis-regulatory element suitable for directing transcription in the subpopulation of cells upon exposure to an agent.

As used herein a “cis acting regulatory element” refers to a naturally occurring or artificial polynucleotide sequence, which binds a trans acting regulator and regulates the transcription of a coding sequence located down-stream thereto. For example, a transcriptional regulatory element can be at least a part of a promoter sequence which is activated by a specific transcriptional regulator or it can be an enhancer which can be adjacent or distant to a promoter sequence and which functions in up regulating the transcription therefrom.

It will be appreciated that the cis-acting regulatory element of the presently preferred embodiment of the invention may be stress regulated (e.g., stress-regulated promoter), which is essentially activated in response to cellular stress produced by exposure of the cell to, for example, chemicals, environmental pollutants, heavy metals, changes in temperature, changes in pH, as well as agents producing oxidative damage, DNA damage, anaerobiosis and changes in nitrate availability or pathogenesis.

Examples of promoters which are preferably used in accordance with the presently preferred embodiment of the invention include, but are not limited to, MipA, LacZ, GrpE, Fiu, MalPQ, oraA, nhoA, recA, otsAB and yciD.

A cis acting regulatory element can also be a translational regulatory sequence element in which case such a sequence can bind a translational regulator, which up regulates translation.

The term “expression” refers to the biosynthesis of a gene product. For example, in the case of the reporter polypeptide, expression involves the transcription of the reporter gene into messenger RNA (mRNA) and the translation of the mRNA into one or more polypeptides.

As used herein “reporter polypeptide” refers to a polypeptide gene product, which, can be quantitated either directly or indirectly. For example, a reporter polypeptide can be an enzyme which when in the presence of a suitable substrate generates chromogenic products. Such enzymes include but are not limited to alkaline phosphatase, β-galactosidase, β-D-glucoronidase (GUS), luciferase and the like. A reporter polypeptide can also be a fluorescer such as the polypeptides belonging to the green fluorescent protein family including the green fluorescent protein, the yellow fluorescent protein, the cyan fluorescent protein and the red fluorescent protein as well as their enhanced derivatives. In such a case, the reporter polypeptide can be quantified via its fluorescence, which is generated upon the application of a suitable excitatory light. Alternatively, a polypeptide label can be an epitope tag, a fairly unique polypeptide sequence to which a specific antibody can bind without substantially cross reacting with other cellular epitopes. Such epitope tags include a Myc tag, a Flag tag, a His tag, a Leucine tag, an IgG tag, a streptavidin tag and the like. Further detail of reporter polypeptides can be found in Misawa et al. (2000) PNAS 97:3062-3066.

It will be appreciated that in certain aspects of the present invention the reporter expression construct may be expressed in response to a growth condition. Examples of such conditions include, but are not limited to temperature, humidity, atmospheric pressure, contact surfaces, radiation exposure (such as, y-radiation, UV radiation, X-radiation).

As mentioned hereinabove, each reporter expression construct is expressed in a subpopulation of cells upon exposure to a distinct agent or groups of agents. It will be appreciated however, that since several unrelated agents can lead to the same effect on a cell, an expression construct can also be expressed albeit at lower effeciency upon exposure to other agents. Such partial overlap between the different reporter expression constructs is desirable since it will increase the detection range of the population to thereby enable identification of numerous agents even at low concentration levels. For example, if a first agent induces reporter expression from one subpopulation it may be difficult to distinguish it from a second unrelated agent which also induces expression in the same subpopulation. However, if several cell subpopulations are induced by a first agent (each subpopulation expressing a unique level of the reporter) the likelihood that the same subpopulations will also react with the same expression pattern upon exposure to a second agent is remote.

Dependent on the host cell used, the reporter expression construct can include additional elements. For example, polyadenylation sequences can also be added to the reporter expression construct in order to increase the translation efficiency of a reporter polypeptide expressed from the expression construct of the present embodiment. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Suitable termination and polyadenylation signals include, without limitation, those derived from SV40.

In addition to the elements already described, the expression construct may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The construct may or may not include a eukaryotic replicon. If a eukaryotic replicon is present, then the vector is amplifiable in eukaryotic cells using the appropriate selectable marker. If the construct does not comprise a eukaryotic replicon, no episomal amplification is possible. Instead, the recombinant DNA integrates into the genome of the engineered cell, where the promoter directs expression of the desired nucleic acid.

The reporter expression construct can be introduced into the cell using a variety of molecular and biochemical methods known in the art. Examples include, but are not limited to, transfection, conjugation, electroporation, calcium phosphate-precipitation, direct microinjection, liposome fusion, viral infection and the like. Selection of a suitable introduction method is dependent upon the host cell and the type of construct used.

Since the response of each subpopulation of the cell population of the presently preferred embodiment of the invention to an agent needs to be assessed independently in order to generate a signature expression pattern, each cell of each subpopulation is preferably tagged with a distinct tag unique to the subpopulation. The tag may be for example, a fluorophoric or chromophoric dye compound which may be detected using a microscope. Such dyes are commercially available such as from Molecular Probes (Eugene, Oreg., USA). Alternatively, cells can be naturally fluorescing or genetically engineered to fluoresce. Molecular tags can also be used. Such tags may be detected by amplification methods, such as PCR.

As stated, system 10 is preferably designed to be stored in a non-operative mode under ambient conditions for prolonged periods of time, whereby selected components of system 10, may be stored under lower temperatures to further extend the shelf life of system 10. Lower storage temperatures are particularly useful when device 11 is removable and/or contains wetware, such as, but not limited to, the aforementioned population and subpopulations of cells, e.g., in a dormant state.

Hence, according to a preferred embodiment of the present invention the sensing device is stored in controlled micro-environmental conditions, so as to maintain the functionality of the sensors therein.

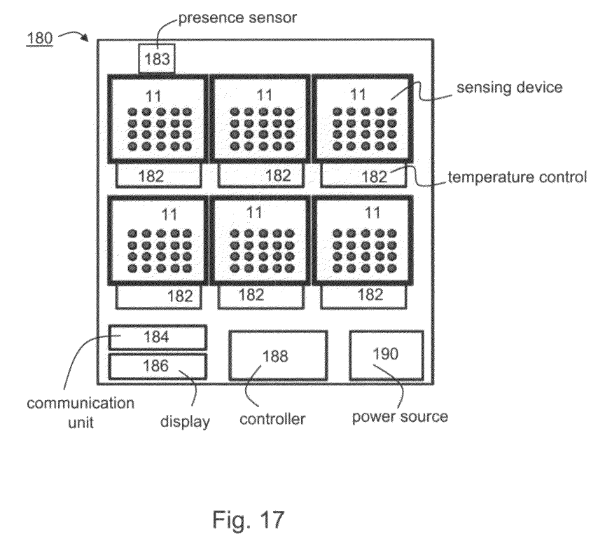

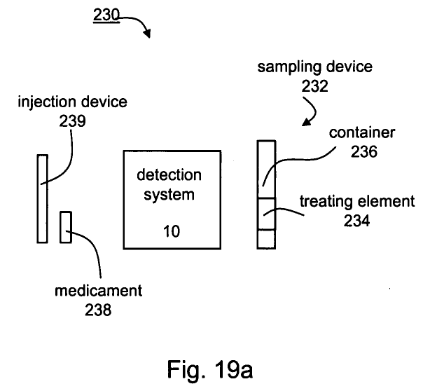

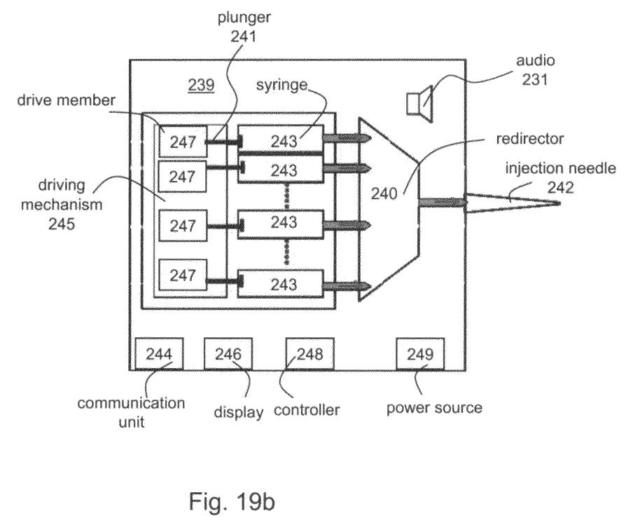

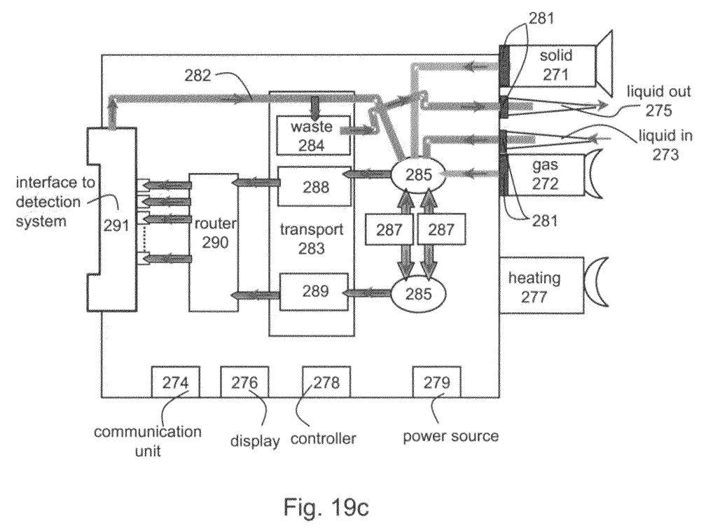

Reference is now made to FIG. 17, which is a schematic illustration of a storage unit 180 for storing sensing device 11, according to a preferred embodiment of the present invention. Shown in FIG. 17 are six sensing devices which can be stored in storage unit 180. It is to be understood that storage unit 180 can be used to store any number of sensing devices, including, without limitation, a single sensing device. Additionally, storage unit 180 can be used to store other object, for example, a sampling device (such as sampling device 270, further detailed herein under with reference to FIGS. 19a and 19c) and collected sample. Thus, while the embodiments below are described with a particular emphasis to the storage of sensing device 11, it is to be understood that more detailed reference to the storage of sensing device 11 is not to be interpreted as limiting the scope of the invention in any way.

Storage unit 180 is preferably portable, so as to allow to users to deliver storage unit to their homes, or any other accessible location. Storage unit preferably comprise a power source 190, which be any type of power source such as, see, e.g., the list of power sources hereinabove, and one or more temperature control units 182 for controlling the temperature of device(s) 11. Any number of temperature control units can be used. Thus, for example, a single temperature control unit can be used to control the temperature of all the sensing devices, or, more preferably, one temperature is allocated to each stored sensing device, such that a thermal communications is established between the temperature control unit the sensing device (or, as stated, any other object stored in storage unit 180).

Temperature control units 182 preferably monitor the temperature of device(s) 11 at all times and ensure and, if necessary, pumps heat off device(s) 11, to thereby provide stable thermal condition. Any type of temperature control unit can be used including, without limitation, thermoelectric device, as further detailed hereinabove.

According to a preferred embodiment of the present invention storage unit 180 comprises a communication unit 184 to allow communication over a communication network with a central unit, other similar storage units and/or any other authorized party connected the communication network. This embodiment is particularly useful when it is desired to remotely (e.g., at a central location) monitor the availability and condition of the sensing devices. To prevent unauthorized parties from monitoring or intervening with the data transfer, communication unit 184 is preferably configured to transmit and receive encoded information. The communication and activation of the various components of storage unit 180 can be controlled by a controller 188.

According to a preferred embodiment of the present invention controller 188, receives temperature information from temperature control units 182 and independently activates each temperature control unit to maintain a predetermined temperature of the sensing device which is in thermal communication therewith. Controller 188 is preferably self-powered (e.g., by an internal power source), and may comprise, for example, RISC, DSP unit and the like. Preferably, controller 188 also receives power level information from power source 190, so as to allow controller 188 to inform the user (e.g., using a display 186) and/or other parties connected to the communicating network when power source 190 is to be recharged or replaced. In addition, controller 188 preferably receives operation status information from temperature control units 182, and informs the user and/or other parties when one or more temperature control units 182 is not operative.

Storage unit 180 may also comprise an arrangement of presence sensors 183 (for simplicity, only one presence sensor is shown in FIG. 17, however any number of presence sensors is contemplated), for determining a number and position of objects occupying storage unit 180. Presence sensors 183 preferably transmit presence and position to controller 188, which in turn independently activates and deactivates the temperature control units, based on this information. The advantage of this embodiment is that only the necessary number of temperature control units is used, so as to reduce energy consumption.

Display 186 preferably displays all relevant information, including without limitation the number of objects stored, the temperature of each object, the status of each temperature control unit, the power level of the power source and the like.

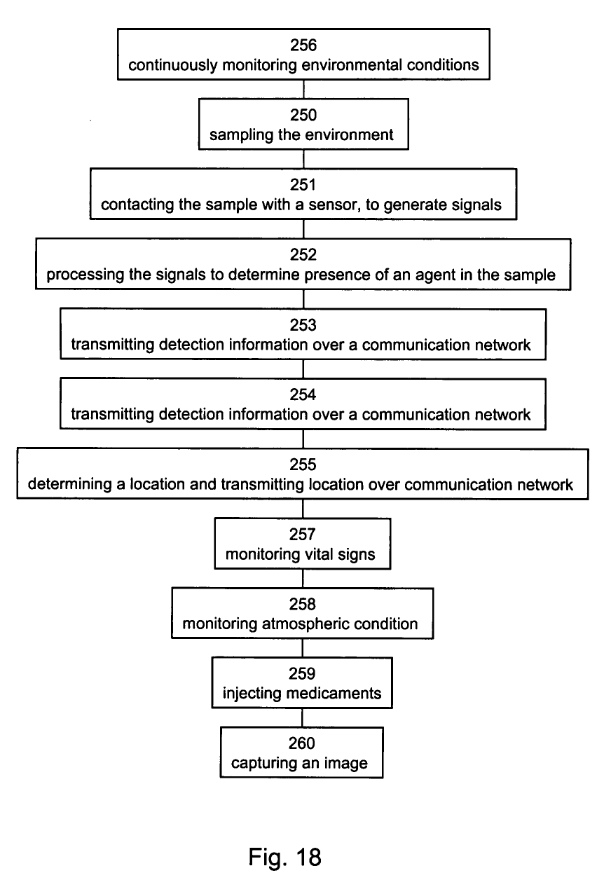

According to another aspect of the present invention there is provided a method of distributing information of a presence of agents in the environment. The method comprises the following method steps which are illustrated in the flowchart of FIG. 18. It is to be understood, that unless otherwise defined, the method steps described hereinbelow can be executed either contemporaneously or sequentially in any combination or order of execution. Specifically, neither the ordering of the flowchart of FIG. 18, nor the numerals designating its various blocks are to be considered as limiting. For example, two or more method steps, appearing in the description or in the flowchart of FIG. 18 in a particular order, can be executed in a different order (e.g., a reverse order) or substantially contemporaneously.

In a first step of the method, designated by Block 250, the environment is sampled to thereby provide a sample. The sample can have any form, such as, but not limited to, fluid, solid, vapor, aerosol and the like. In another step of the method, designated by Block 251, the sample is delivered through a plurality of channels (e.g., channels 14 of device 11) so as to contact the sample a sensor (e.g., sensor 18). Upon exposure of the sensor to an agent in the sample, a signal (optical, electrical, electrochemical, etc.) is generated. In an additional step, designated by Block 252 the signals generated by the sensor are processed, for example, using a data processor, so as to determine the presence or level of the agent in sample. In an additional step, designated by Block 253 the detection information (e.g., presence, absence, level, type) of the agent is transmitted over the communication network.

According to a preferred embodiment of the present invention the method comprises an additional step, designated by Block 255, in which a location, at which the sampling takes place, is obtained and transmitted over the communication network. The location can be determined using any of the aforementioned positioning techniques.

In another optional step of the method, designated by Block 256, environmental conditions are continuously monitored. This step is preferably executed either contemporaneously with any other step of the method or in predetermined instances or time intervals. According to a preferred embodiment of the present invention the monitoring of the environmental conditions is used to trigger the sampling of the environment. For example, when environmental conditions meet a predetermined criterion, e.g., preliminary detection of a potentially harmful agent, the sampling is performed. Additionally, vital signs of a mammal may also be measured (Block 257), for example, for triggering the sampling by an appropriate criterion (e.g., reduction in vital signs) and/or transmission of the vital signs over the communication network.

In a further step of the method, designated by Block 258, atmospheric condition can also be monitored and transmitted over the communication network. As stated, this information can be used to predict a propagation path of the detected agent.

Additional steps of the method include injection of one or more medicaments in case of a contact between a subject and the agent (Block 259) and capturing an image of the environment or portion thereof (Block 260), which image can then be transmitting over the communication network.

Selected steps of the above method can be performed by system 10. Other steps can be performed by suitable devices which can be supplied as accessories to system 10. These devices and system 10 can be incorporated in a detection kit which can be released into the population of interest during or prior to initiation of a diffuse signal, for example, in a form of a harmful agent. Hence, according to another aspect of the present invention there is provided a portable detection kit 230, for detecting agents present in a sample.

Reference is now made to FIG. 19a, which is a schematic illustration of kit 230. Kit 230 preferably comprises one or more sampling devices 270, for selectively sampling environmental material, and a portable detection system, which can be similar to system 10 or a variant thereof. In use, sampling device 270 samples the environmental materials and feeds the sampled materials into system 10 which senses and analyses the materials as further detailed hereinabove. The materials sampled by sampling device 270, or a portion thereof, can also be collected for further analysis in an appropriate facility, e.g., a microbiology laboratory. Optionally and preferably, sampling device 270 treat the sampled material prior to the feeding into system 10 to enhance the detection accuracy, as further detailed hereinunder.

According to a preferred embodiment of the present invention kit 230 can comprise at least one medicament 238 and optionally an medical injection device 239 for injecting medicament 238. Medicament(s) 238 are preferably selected according to the expected threat so as to allow self-treatment of the user in case of injury. Medical injection device 239 is preferably easy to carry and safe to use. More particularly, injection device 239 is preferably useful for carrying medicaments such as atropine, lidocaine, heart medication, allergy medication and the like. Injection devices are known in the art and found, e.g., in U.S. Pat. Nos. 6,530,904, 6,758,110 and 5,968,015, and U.S. Patent Application Nos. 20040134563, 20040158205, 20040064041.

More preferably, injection device 239 is designed to contain more than one medicament and to select, preferably automatically upon receiving an appropriate signal, a medicament which is most suitable to the type of the detected, or expected to be detected, agent.

Reference is now made to FIG. 19b, which is a schematic illustration of medical injection device 239, according to a preferred embodiment of the present invention.

Injection device 239 preferably comprises an injection needle 242 and a plurality of syringes 243, each having a plunger 241, which slides within the respective syringe. Each syringe preferably contains different medicament. Injection device 239 further comprises a driving mechanism 245 having a plurality of drive members 247. Each drive member is design to engage a plunger of one syringe. Injection device 239 further comprises a redirector 240 for redirecting medicaments, released by one or more syringes, into injection needle 242.

Injection device 239 is preferably supplemented with a power source 249 which supplies power to the various units of device 239. According to a preferred embodiment of the present invention, injection device 239 further comprises a communication unit 244, which can receive signals via a communication network, which signals are specific to the type and does of the suitable medicament. To prevent unauthorized parties from monitoring or intervening with the data transfer, communication unit 244 is preferably configured to transmit and receive encoded information. Injection device 239 may also comprise a display 246 and/or an audio device 231 for displaying and/or playing information to the user.

In use, driving mechanism 245 receives activation signals from communication unit 244, e.g., via a controller 248, and activate the appropriate syringe or syringes, which intern extrude the desired dose of medicament(s) into redirector 240. The medicament(s), are then redirected into needle 242 and injected into the subject thereby.

Sampling device 270 is preferably designed and constructed to be used more than one time, more preferably a plurality of times. According to a preferred embodiment of the present invention sampling device is adapted to sample environmental fluids (gases or liquids), or solids (e.g., particulates and the like). Thus, sampling device may be provided in a form of a syringe having a syringe needle, which can be operated, as commonly known in the art, to sample materials from the environment by forming an under-pressure within the syringe relative to the environmental pressure.

Sampling device 270 can be adapted either to a continuous sampling or to a single-batch sampling. A particular feature of the present embodiment is the ability of sampling device 270 to treat the sampled materials prior to the feeding into detector 30. Hence, sampling device 270, preferably comprises container 236 and at least one treating element 234 for treating said environmental materials in container 236. Many treating element are contemplated, including, without limitation a filter, an enriching unit, an elution unit, a heating unit, an irradiation unit, a labeling unit, a separating column a sorter, a biological material and the like.

Thus, treating element(s) 234 can be used to perform many treatments which preferably optimize the detection performance of system 10 in terms of speed, specifity and reliability. The treatments can be chemical and/or physical treatments, including, without limitation, screening, filtration, adsorption, desorption, elution, concentration, chemical reactions, heating and labeling. The treatment can also be used to ensure that the sample is delivered to system 10 at an appropriate phase, for example, airborne gas, liquid-dissolved gases, liquid-suspended particulates and the like.

Reference is now made to FIG. 19c, which is a schematic illustration of a preferred configuration of sampling device 270, according to another embodiment of the present invention. In this embodiment, sampling device 270 preferably comprises a communication unit 274, for communicating with a central unit, or communication unit 13 of system 30. Additionally, communication unit 274 can communicate with other similar sampling devices or any other appropriate and authorized party. To prevent unauthorized parties from monitoring or intervening with the data transfer, communication unit 274 is preferably configured to transmit and receive encoded information. The communication and operation of communication unit 274 as well as other components of sampling device 270, further detailed below, is preferably controlled by a controller 278, which may comprise, for example, RISC, a DSP unit and the like. Sampling device 270 can also comprises a display 276 for displaying information, for example, of the amount, phase or speed of sample acquisition, or any other relevant information, which is provided by controller 278. Sampling device preferably comprises a power source 279, of any type as further detailed hereinabove.

According to a preferred embodiment of the present invention sampling device comprises an interface 291 compatible (shapewise and sizewise) with one or more of the sample ports of device 11 (not shown, see, e.g., FIG. 1b) so as to allow sealed fluid communication therebetween. Interface 291 is preferably configured to facilitate fast connection and detachment between system 10 and sampling device 270.

Sampling device 270 preferably comprises several acquisition units, designated in FIG. 19c by numerals 271, 272 and 273, to enable separate acquisition of solid (271), gas (272) and liquid (273). In case of solid and gas the acquisition units are preferably capable of binding the sampled material to a liquid phase.

For example, for gaseous samples, the binding can be done by stripping the air sample through a liquid medium and/or adsorption onto a solid medium or surface followed by a controlled (e.g., temperature mediated) release. For solid samples, the binding can be done by mixing with a liquid medium.

One or more of the acquisition unit may comprise a suitable treating element, e.g., a filter 281, for selective sampling, e.g., of molecules or suspended particles below a certain size. The filter may be any type of filter, such as, but not limited to, a membrane, a paper, a polymer, a glass and the like. Additionally, the suspended particles can be bind to a liquid phase for further analysis. This embodiment is particularly useful for analysis of bacteria, fungi, viruses or protozoa in spore or non-spore states. Additionally, sampling device 270 preferably comprises a heating element 277, which can be used, for example, for heating surfaces prior to sampling hence to improve the sampling efficiency.

According to a preferred embodiment of the present invention sampling device 270 comprises a plurality of channels 282 connecting the acquisition units with other components of sampling device. Sampling device 270 may further comprises a one or more containers 287 connected by channels 282 to one or more mixers 285, for treating the sampled materials by mixing them, for example, with clean water or appropriate nutrition. One or more containers can also contain concentrated buffer at a desired pH value (for example, Tris buffer, 1M, pH 7, 1% by volume) so as to maintain the desired pH value of the sample.

Mixers 285 are also in fluid communication to one or more containers from which the liquid is transferred, via a router 290, to the various sample ports of system 10 for detection. The flow of liquid through the various channels of sampling device 270 is preferably actuated by a transport mechanism, which can be of any type as further detailed hereinabove.

Any number of containers can be used. For example, in the embodiment of FIG. 19c, two containers are employed: a first container 288 for holding control liquids and a second container 289, for holding sample liquids. The liquids from each container are preferably transferred to different reaction chamber or different logical or physical divisions of sensing device 11.

The fluid communication between sampling device 270 and sensing device 11 is preferably bilateral, so as to allow draining of liquids out of sensing device 11. Preferably, waste liquids are transferred to a waste container 284, from which they are drained out of sampling device 270 through a drainer 275. Liquids exiting sensing device may also routed to one or more of mixers 285, if desired.

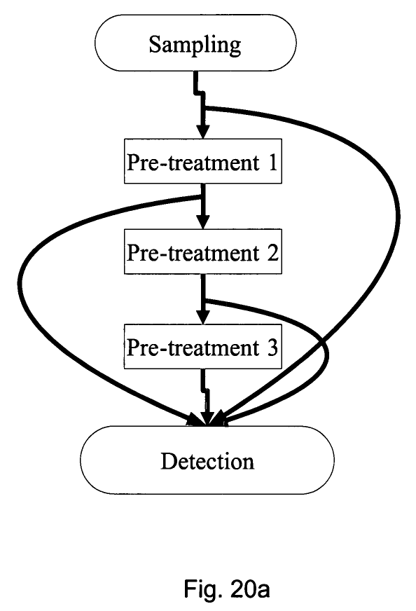

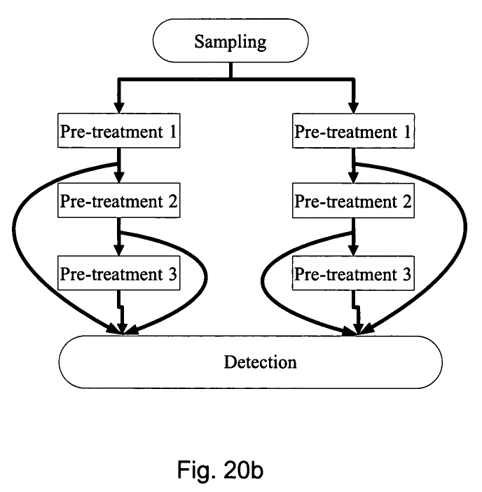

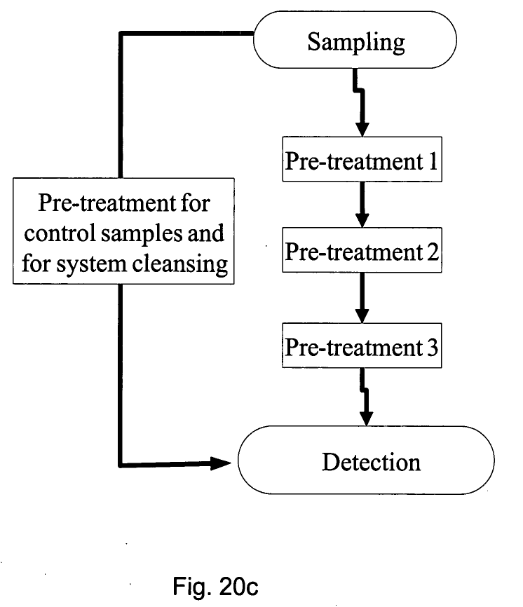

Reference is now made to FIGS. 20a-c, which are flowchart diagrams of several general treatment scenarios which can be employed.

Hence, According to a preferred embodiment of the present invention sampling device 270 can perform more than one treatment, either sequentially (FIG. 20a) or contemporaneously (FIG. 20b) as desired. Additionally, sampling device 270 can perform the treatment(s) on a portion or the entire samples and to perform different treatments to different portions of the sample. According to a preferred embodiment of the present invention the treated sample or portion thereof can be transferred to system 10 in any steps of the treating process, if desired. This embodiment can be used, for example, to skip one or more of the treating steps or to compare detection results obtained after different number of treating steps (see FIG. 20c).

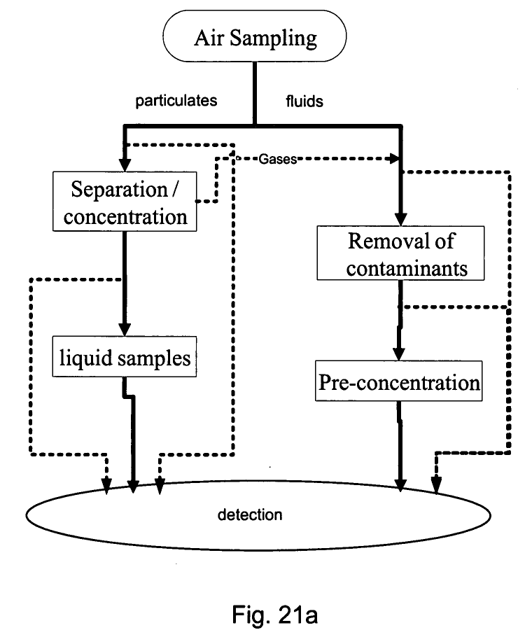

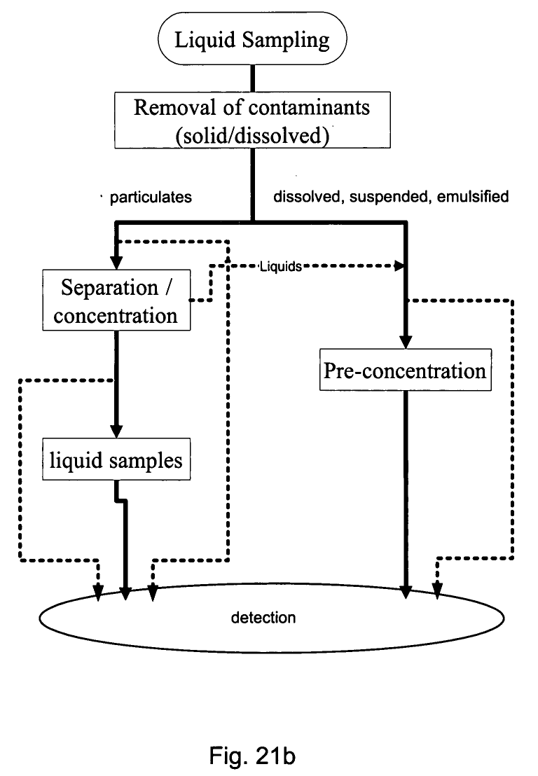

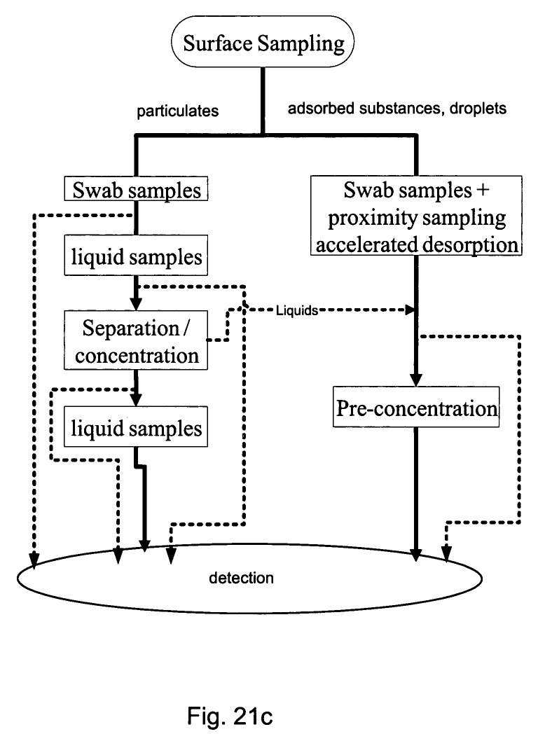

Reference is now made to FIGS. 21a-c, which are flowchart diagrams of treatment scenarios which can be employed, for sampling of air (FIG. 21a), liquid (FIG. 21b) and surface (FIG. 21c), according to a preferred embodiment of the present invention.

When kit 230 is used for air sampling (see FIG. 21a) for airborne toxic chemicals, contemporaneous treatments are preferably performed on particulates (typically biological and radioactive agents) and fluids (typically chemical and radioactive agents). Large volumes of air may be passed through conduits tailored to specifically adsorb target chemicals (for example by using appropriate polymer matrixes). The chemicals can then be desorbed from the adsorbing matrix (e.g., by the application of heat) so as to provide system 10 with an enriched sample. Similarly, when kit 230 is used for monitoring air for the presence of potentially pathogenic airborne particles, the particles may be concentrated by means of passing the sampled a air through impactors (cascade or virtual), cyclones and the like, which provide an enriched sample. The particles can be directly analyzed by device 11 or, more preferably, transferred to small volumes of liquids for further analysis. The dislodging of particles from the impactors may be achieved by electrostatic, acoustic or any other appropriate method.

When kit 230 is used for liquids sampling (see FIG. 21b), a removal of solid or dissolved contaminates is preferably followed by contemporaneous treatments performed on particulates (typically biological and radioactive agents) and dissolved, suspended or emulsified substances (typically chemical and radioactive agents).

When kit 230 is used for surface sampling (see FIG. 21c), contemporaneous treatments are preferably performed on particulates (typically biological and radioactive agents) and adsorbed substances and droplets (typically chemical and radioactive agents).

For biological materials, the treatments preferably comprise one or more nucleic acid amplification procedures, such as, but not limited to, polymerase chain reaction (PCR), ligase chain reaction (LCR), strand displacement amplification (SDA) and self-sustained sequence replication (3SR). In this embodiment, a number of additional steps may be implemented, for example, lysis of target cells by appropriate solutes, purification of the lysates, addition of reagents and application of temperature cycles.

The kit of the present embodiment may, if desired, be presented in a pack or dispenser device, such as an FDA approved kit, which may contain one or more units of the kit of the present embodiment. The pack may be accompanied by instructions for use. The pack may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or food supplements of an approved product insert.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non limiting fashion.

### Example 1

CMOS Detector

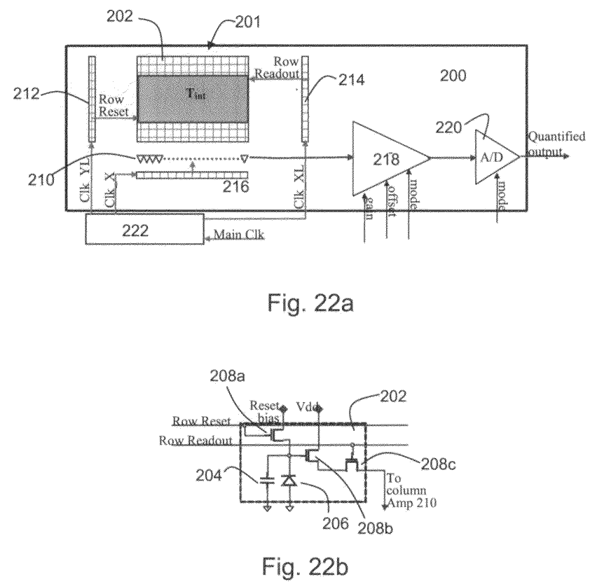

Reference is now made to FIG. 22a-b exemplifying an electronic diagram of a CMOS detector 200, which can be used as detector 108, according to a preferred embodiment of the present invention. CMOS detector 200 is known in the art and can be purchased, for example, from Fill Factory, Mechelen Belgium.

CMOS detector 200 comprises a matrix 201 of elementary units 202 referred to herein as pixels 202. FIG. 22b shows one pixel 202, which comprises a capacitor 204 which is pre-charged to a reset bias voltage, a photodiode 206, for discharging capacitor 204 in response to photons absorption and 3 MOS transistors, designated 208a, 208b and 208c, for resetting (transistor 208a), sensing (transistor 208b) and leading (transistor 208c) the signal to column amplifier 210.

CMOS detector 200 further comprising a left vertical shift register 212 and a right vertical shift register 214. Left register 212 serves a pointer to a row that is pre-charged to reset bias voltage (reset row operation) and right register 214 serves as a pointer to a row which is being read by column amplifier 210. The distance between two pointers determines the integration time, Tᵢₙₜ, which can be calculated as follows:

Tᵢₙₜ=N_{int-rows}·(T_{rd-px}·N_{X}+T_{blanking}), (EQ. 1)

where N_{int-rows} is the distance in rows between the readout and the reset row pointers, T_{rd-px} is the time for one pixel readout, N_{X} is the size of the window in X-direction and the T_{blanking} is the minimal required time between two successive row readouts. T_{rd-px} and T_{blanking} depend on signals generated in a clock unit 222.

CMOS detector 200 further comprises a column shift register 216 which selects the appropriate column amplifier 210. The signal from column amplifier 210 is transmitted through a pre-amplifier 218 to a digital converter 220.

Pre-amplifier 218 and converter 220 preferably operate in linear mode. The quantified output of CMOS detector 200 is proportional to the number of photons colliding on pixels 202, denoted Nₚₕₜ:

$${N}_{pht} ( {T}_{int} ) = C \frac{( QO ( {T}_{int} ) - {QO}_{DC\_e -} ( {T}_{int} ) ) / {2}^{bits}}{QE \cdot FF} , \qquad ( EQ . 2 )$$

where QO is a quantified output, bits is the quantization of converter 220, QE is a quantum efficiency of the CMOS detector 200, FF is a fill-factor, C is a maximal capacity of pixel in photo-electrons and QO_{DC}_{_{—}}ₑ₋ is a quantified output of an integrated dark current, I_{DC}.

The time dependence of QO_{DC}_{_{—}}ₑ₋ is given by:

$${QO}_{DC\_e -} ( T ) = \frac{{I}_{DC}}{C} T \cdot {2}^{bits} , \qquad ( EQ . 3 )$$

where the units of I_{DC}, C and T are such that the expression I_{DC}T/C is dimensionless. For example, I_{DC}, C and T can be measured in e⁻/s, e⁻ and s, respectively.

For the CMOS detector of the present example, a typical value of C, is 6×10⁴. The value of the dark current, I_{DC}, is about 1055[e⁻/s], for a temperature of 21° C.

The required integration time is inversely proportional to the intensity of the signals. On the other hand, this maximal integration time is limited because of dark current saturation effect.

For weak signals, the maximal integration time is given by:

$${T}_{int \text{-} max \text{-} ws} = \frac{C}{{I}_{DC}} \cdot {F}_{DC} \qquad ( EQ . 4 )$$

where F_{DC} is a parameter defined by the following equation:

QO_{DC}_{_{—}}ₑ₋_{_{—}}ₘₐₓ=F_{DC}·2^{bits}. (EQ. 5)

A typical value of F_{DC} is about 0.5.

For strong signals, the maximal integration time is given by:

$${T}_{int \text{-} max \text{-} ss} = \frac{C}{{I}_{s} \cdot QE \cdot FF} , \qquad ( EQ . 6 )$$

where Iₛ is the strong signal current.

### Example 2

### Determination of Agent Concentration Using the Detected Signals

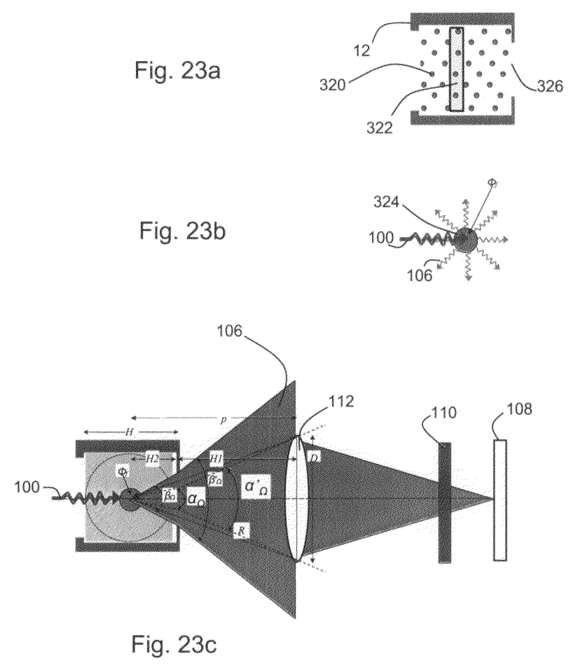

Reference is now made to FIGS. 23a-c, which illustrate the radiation emitted by one reaction chamber of system 10.

FIG. 23a illustrates reaction chamber 12, a plurality of locations 320, where biological cells generating the florescent materials are located, and a slice 322. Reaction chamber 12 has an aperture 326 through which optical signals 106 (not shown, see FIGS. 23b-c) exit. In the following calculations, slice 322 is represented as equivalent light emitter 324, shown in FIG. 23b. Equivalent light emitter 324 is a superposition of the all the light emitter in slice 322 and can be defined, for example, by integration or summation. Also shown is excitation light 100 and optical signal 106 emitted in a plurality of directions.

FIG. 23c illustrates the spreading of optical signal 106 through aperture 326 of the reaction chamber. The respective numerical aperture for optical signal 106, designated herein by β′_{Ω}, depends on the position of slice 322. Optical signal 106 is collimated by lens 112 prior to impingement of optical signal 106 on light detector 108. Being spaced apart from aperture 326 the corresponding numerical aperture of lens 112, designated herein by α′_{Ω}, is smaller than the numerical aperture of aperture 326, β′_{Ω}. Similarly to β′_{Ω}, α′_{Ω} also depends on the position of slice 322. Emission filter 110 is positioned in the light path of optical signal 106 so as to prevent rays of excitation light 100 from arriving to detector 108. Emission filter 110 preferably allows transmission of optical signal 106 substantially without loses.

The use of lens 112 is preferred, but optional. In an alternative embodiment in which lens 112 is not used, emission filter 110 and detector 108 are preferably positioned instead of lens 112, with no substantially change in the values of the β′_{Ω} and α′_{Ω}.

According to a preferred embodiment of the present invention several optical coefficients are calculated. A first optical coefficient is the emission quantum efficiency, denoted herein by EmQE, which is the ratio between the absorbed excitation radiation and the emitted radiation. A second optical coefficient is the transmission coefficient of emission filter 110 for excitation light 100, designated herein by T_{Fem4exc}. A third optical coefficient is the transmission coefficient of emission filter 110 for optical signal 106, designated herein by T_{Fem4ems}. A fourth optical parameter is the effective quantum efficiency of detector 108 for optical signal 106, designated herein by QEₑₘₛ, and a fifth optical parameter is the effective quantum efficiency of detector 108 for excitation light 100, designated QE_{exc}.

The value of the above five coefficients depends on the spectral characteristics of the optical components. More specifically, the following spectra are used for the calculations: (i) the spectrum of the light source, SL; (ii) the spectrum of excitation filter SF_{exc} (iv) The spectrum of emission filter 110, S_{Fem}; (v) the efficiency of the fluorescence excitation E_{fl}; (vi) the emission spectrum, Sₑₘₛ; and (vii) the Quantum Efficiency (QE) of light detector 108.

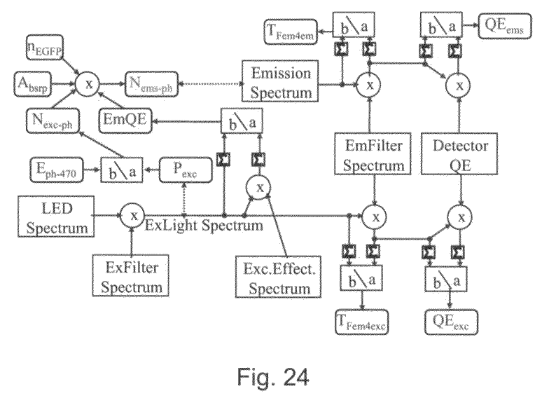

The calculation of the optical coefficient can be done using the following formulae:

$$\begin{matrix}EmQE = \frac{{\sum }_{λ} {S}_{L} {S}_{Fexc} {E}_{fl}}{{\sum }_{λ} {S}_{L} {S}_{Fexc}} , & ( EQ . 7 a ) \\ {T}_{Fem 4 exc} = \frac{{\sum }_{λ} {S}_{L} {S}_{Fexc} {E}_{Fems}}{{\sum }_{λ} {S}_{L} {S}_{Fexc}} , & ( EQ . 7 b ) \\ {T}_{Fem 4 ems} = \frac{{\sum }_{λ} {S}_{ems} {S}_{Fems}}{{\sum }_{λ} {S}_{ems}} , & ( EQ . 7 c ) \\ {QE}_{ems} = \frac{{\sum }_{λ} {S}_{ems} {S}_{Fems} QE}{{\sum }_{λ} {S}_{ems} {S}_{Fems}} , & ( EQ . 7 d ) \\ {QE}_{exc} = \frac{{\sum }_{λ} {S}_{L} {S}_{Fexc} {S}_{Fems} QE}{{\sum }_{λ} {S}_{L} {S}_{Fexc} {S}_{Fems}} , & ( EQ . 7 e )\end{matrix}$$

where A_{bsrp} is the ratio between the area of equivalent light emitter 324 to the area of the excitation beam, n_{GFP}, is the occupation of the fluorescent material, measured as a percentage of the area of equivalent light emitter 324, Nₑₘₛ₋ₚₕ is a number of the emission photons per second, N_{exc-ph} is a number of the excitation photons per second, Eₚₕ₋₄₇₀ is energy of single photon at the wavelength 470 nm and P_{exc} is the optical power. FIG. 24 is a schematic calculation diagram which can be implemented for the calculation of Equations 7a-7e.

According to a preferred embodiment of the present invention once the optical coefficients are calculated, a ray tracing procedure is employed.

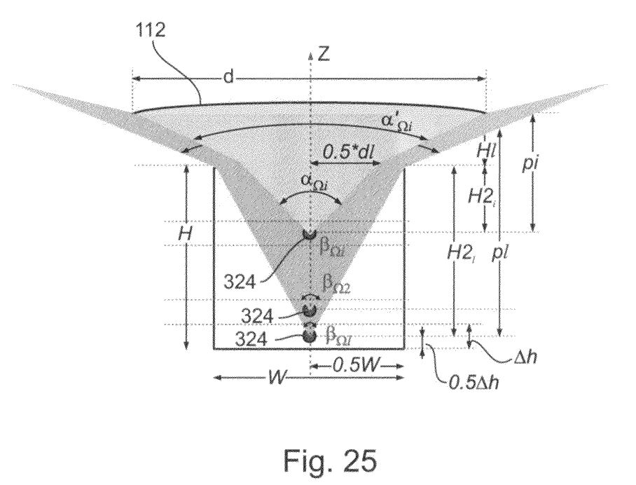

FIG. 25 illustrates light propagation from equivalent light emitter 324 to lens 122. Light rays are redirected to the angle β′_{Ω}, according to Snell's law

n_{RC}=sin(β_{Ω}/2)=n·sin(β′_{Ω}/2) (EQ. 8)

where n_{RC} is the refraction index of the medium in reaction chamber 12 and n is the refraction index of the external medium.

Assuming that the there are B_{cube} biological reporters in a 1 mm³ cube, the number of biological reporters in one dimension B_{1D}, is the cubic root of B_{cube}. Thus, denoting the height of reaction chamber by H, the total number of slices is:

N_{layers}=B_{1D-1mm}·(H/1 mm) (EQ. 9)

Thus, each slice has a thickness of:

Δh=H/N_{layers}, (EQ. 10)

and is centered at position H₂ᵢ, where:

H₂ᵢ=H−Δh·(i−0.5). (EQ. 11)

The scattering angle of optical signal from the ith slice is given by:

β_{Ωi}=2·tg⁻¹(W/2H₂ᵢ), (EQ. 12)

where W is the diameter of aperture 326.

Using Equations 8 and 12 one can calculate the numerical aperture, β′_{Ω}:

β′_{Ω}=2·sin⁻¹((n_{RC}/n)·sin(tg⁻¹(W/2H₂ᵢ))). (EQ. 13)

The numerical aperture of the lens for the ith slice is given by:

α_{Ωi}=2·tg⁻¹(dl/2H2ᵢ), (EQ. 14)

where H₂ᵢ is the distance between the center of the ith slice and output aperture and dl is twice the distance between the optical axis of the lens and the emitted light ray (see FIG. 25), which can be numerically calculated, for example, by an iterative procedure.

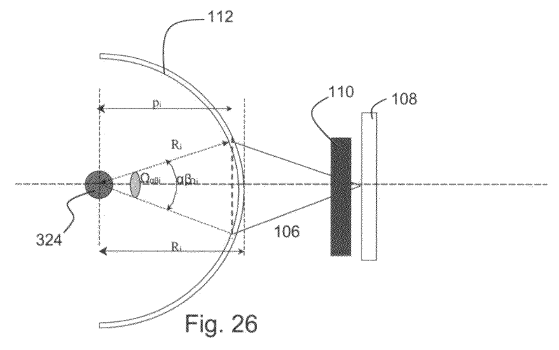

FIG. 26 illustrates the scattering solid angle of the emitted light rays. Hence, defining the range αβ_{Ωi} as min(α_{Ωi}, β_{Ωi}), light rays emitted from the ith slice at an angle within range αβ_{Ωi}, are redirected to detector 108.

Generally, rays 106 are scattered uniformly to a solid angle of 4π. The fraction of emission energy impinging on detector 108 is therefore:

$${F}_{GFP} = \frac{{Ω}_{a β i}}{4 π} , \qquad ( EQ . 14 )$$

where Ω_{αβi} is the solid angle corresponding to range αβ_{Ωi} and is given by:

Ω_{αβi}=2π(1−cos(0.5αβ_{Ωi})). (EQ. 15)

Following is a description of a calculation of the absorption of the excitation light and the corresponding emission of optical signals 106.

A photon having wavelength λ carries energy which equals:

$${E}_{ph} ( λ ) = \frac{h \cdot c}{λ} . \qquad ( EQ . 16 )$$

For example, when the excitation wavelength is 470 nm, the energy carried by one excitation photon is 3.810⁻¹⁹ J. Assuming that the excitation light is transmitted by an optical fiber, photon flux (the number of photon per unit time) is given by:

$${I}_{exc} = \frac{{P}_{fiber}}{{E}_{ph} ( {λ}_{exc} )} , \qquad ( EQ . 17 )$$

where P_{fiber} is the optical power of the optical fiber which can be measured, for example, using an optical power meter positioned on the output of the optical fiber.

The amount of optical signals generated by the biological material is proportional to the projection area of the fluorescent material on a plane perpendicular to the direction defined by detector 108 and slice 324.

For a d_{b}×d_{b}×d_{b} cube, the maximum absorption by the fluorescent material of the ith slice is:

$${A}_{GFP - max - i} = \frac{{d}_{b}^{2} \cdot {B}_{layer}}{π \cdot {( W / 2 )}^{2}} , \qquad ( EQ . 18 )$$

where B_{layer} is the number biological reporters in ith slice:

$${B}_{layer} = \frac{{B}_{RC}}{{N}_{layers}} . \qquad ( EQ . 19 )$$

The maximal absorption occurs for when the biological material is completely saturated by the fluorescent material. The efficiency of the bio-chemical reaction is proportional to the percentage of the biosensor saturation by the GFP molecules. The percentage of the saturation in layer i is signed as n_{GFP-i}.

The light absorption in the ith layer is given by:

A_{GFP-i}=A_{GFP-max-i}·n_{GFP}, (EQ. 20)

where A_{GFP-max-i} is the maximal absorption.

The value of the n_{GFP} can be between zero and unity inclusive. n_{GFP}=0 means that is that no fluorescent material was generated, while n_{GFP}=1 means that the entire biological cell is saturated by fluorescent material and the absorption equals A_{GFP-max-i}.

Assuming that the dominant attenuation of the excitation light is due to the fluid in the reaction chamber, the absorbed excitation light in the ith slice is given by:

I_{exc-i}=I_{exc-i-1}·T_{exc}(Δh)=I_{exc}·T_{exc}ⁱ⁻¹(Δh). (EQ. 21)

The maximal intensity of the emitted optical signal, scattered at a solid angle of 4π, can be written as:

Iₑₘₛ₋ₘₐₓ₋ᵢ=A_{GFP-max-i}·I_{exc-i}·EmQE, (EQ. 22)

and the optical signal intensity as function of the biochemical reaction efficiency is therefore given by:

Iₑₘₛ₋ᵢ=Iₑₘₛ₋ₘₐₓ₋ᵢ·n_{GFP} (EQ. 23)

Integrating over all slices one thus obtain a relation between the detected signal and n_{GFP}. One ordinarily skilled in the art would appreciate that from the value of n_{GFP} the concentration of the agent can be obtained, for example, using a simple calibration curve.

### Example 3

### Sensitivity Calculation

As stated, the emission intensity is proportional to the biochemical reaction percentage expressed by the n_{GFP} parameter. In the present example, a sensitivity calculation is performed using a signal uncertainty parameter, which is proportional to the unfiltered excitation intensity detected by the light detector:

I_{unc-px}=Un2B·I_{exc-px}·QE_{exc}, (EQ. 24)

where I_{unc-px} is the signal uncertainty parameter, Un2B is the ratio between the uncertainty to the background radiation, Iₑₘₛ₋ₚₓ is the unfiltered excitation intensity as detected by the light detector and QE_{exc} is, as stated, the effective quantum efficiency of the detector for excitation light. A tipical value for Un2B is about 0.5.

The minimal sensitivity is preferably defined such that the sensed optical signal is at least S2Un times stronger that the signal uncertainty, where S2Un is the ratio between the signal to the uncertainty:

Iₑₘₛ₋ₚₓ·QEₑₘₛ≧S2Un·I_{unc-px}, (EQ. 25)

where Iₑₘₛ₋ₚₓ is the emission intensity as detected by the light detector, and QE_{exc} is the effective quantum efficiency of the detector for emission light. The emission intensity from the biological sensor is proportional to the n_{GFP}, thus:

R_{ems/exc}=R_{ems/exc-max}·n_{GFP}

Iₑₘₛ₋ₚₓ=Iₑₘₛ₋ₘₐₓ₋ₚₓ·n_{GFP}, (EQ.26)

where, R_{ems/ex} is the ratio between emission and excitation, R_{ems/ex-max} is the maximal ratio between emission and excitation and Iₑₘₛ₋ₚₓ is the maximal emission intensity which can be detected.

Combining the Equations 24-26 one has:

$$\frac{{I}_{ems \text{-} max \text{-} px} \cdot {QE}_{ems}}{{I}_{exc - px} \cdot {QE}_{exc}} \cdot {n}_{GFP} \geq S 2 Un \cdot Un 2 B , \qquad ( EQ . 27 )$$

### Denoting:

$${R}_{ems / exc \text{-} max} = \frac{{I}_{ems \text{-} max \text{-} px} \cdot {QE}_{ems}}{{I}_{exc - px} \cdot {QE}_{exc}} , \qquad ( EQ . 28 )$$

Equation 27 can be written as:

R_{ems/exc-max}·n_{GFP}≧S2Un·Un2B. (EQ. 29)

The constant R_{ems/exc-max} depends on the optical setup, and can be determined, experimentally, or by geometrical optic calculations. From Equation 29 it follows that the minimal optical system sensitivity to the bio-chemical reaction is:

$${n}_{GFP - min} = \frac{S 2 Un \cdot Un 2 B}{{R}_{ems / exc \text{-} max}} . \qquad ( 30 )$$

### Example 4

### Biological Signal Quantification

In this example, the biological signal quantification is demonstrated for a system in which excitation energy is transmitted using external optical fibers and the sensors are E. coli bacteria, capable of producing GFP molecules when interacting with a toxic material. The bacteria were placed in the reaction chamber in the presence of a Luria Bertani nutrition medium.

The detected signal, QO_{int-sig}, is a sum of the following contributions (i) unfiltered excitation intensity, I_{exc-Fem4ex}; (ii) intensity of emission generated by nutrient medium, I_{LB}; (iii) intensity of light emitted by the GFP molecules, I_{GFP}; and (iv) dark current contribution of the light detector, I_{DC}.

The overall emission intensity that reaches the pixels of the detector is given by:

$${I}_{ems - mlens - det} = {\sum }_{i = 1}^{i = {N}_{layers}} ( {A}_{GFP - max} \cdot {I}_{exc - i} \cdot {n}_{GFP} \cdot {T}_{ems} ( {H}_{2 i} ) \cdot \frac{{Ω}_{a β i}}{4 π} \cdot {T}_{mlens} \cdot {T}_{Fems 4 ems )} \qquad ( EQ . 31 )$$

For an excitation wavelength of 470 nm, the excitation at the ith slice is:

$$\begin{matrix}{I}_{exc - i} = ( \frac{{P}_{fiber}}{{E}_{ph} ( 470 nm )} ) \cdot {T}_{exc}^{i - 1} ( Δ h ) \\ = {P}_{fiber} \cdot ( \frac{{T}_{exc}^{i - 1} ( Δ h )}{{E}_{ph} ( 470 nm )} ) .\end{matrix} \qquad ( EQ . 32 )$$

The light intensity, emitted by the GFP molecules is therefore given by:

$${I}_{ems - mlens - det} == {\sum }_{i = 1}^{i = {N}_{layers}} ( {A}_{GFP - max} \cdot {P}_{fiber} \cdot ( \frac{{T}_{exc}^{i - 1} ( Δ h )}{{E}_{ph} ( 470 nm )} ) . {n}_{GFP} \cdot {T}_{ems} ( {H}_{2 i} ) \cdot \frac{{Ω}_{a β i}}{4 π} \cdot {T}_{mlens} \cdot {T}_{Fems 4 ems} ) == {n}_{GFP} \cdot {P}_{fiber} \cdot {{\sum }_{i = 1}^{i = {N}_{layers}} {( {A}_{GFP - max} \cdot ( \frac{{T}_{exc}^{i - 1} ( Δ h )}{{E}_{ph} ( 470 nm )} ) \cdot {T}_{ems} ( {H}_{2 i} ) \cdot \frac{{Ω}_{a β i}}{4 π} \cdot {T}_{mlens} \cdot {T}_{Fems 4 ems} )}_{1 \; 4 \; 4 \; 4 \; 4 \; 4 \; 4 \; 4 \; 4 \; 4 \; 4 \; 4 \; 44 \; 2 \; 4 \; 4 \; 4 \; 4 \; 4 \; 4 \; 4 \; 4 \; 4 \; 4 \; 4 \; 4 \; 43}}_{{K}_{bs - exe - ems - optics - mlens}} \qquad ( EQ . 33 )$$

where K_{bs-exc-ems-optics-mlens} is a constant coefficient depending on the geometry of the reaction chamber, the concentration of bacteria, the optical characteristics of the optical components (filters and lenses) and the photon energy. The emission intensity which achieves the light detector is given by:

I_{ems-mlens-det}=n_{GFP}P_{fiber}·K_{bs-exc-ems-optics-mlens} (EQ. 34)

Denoting the area onto which the optical signal is spread on the detector by S_{ems-det}, and the area of one pixel by Sₚₓ, the emission intensity projected on one pixel is:

Iₑₘₛ₋ₘₗₑₙₛ₋ₚₓ=n_{GFP}·P_{fiber}·K_{bs-exc-ems-opics-mlens}·(Sₚₓ/S_{ems-det})=n_{GFP}P_{fiber}·K_{bs-px} (EQ. 35)

where, K_{bs-px} is a numerical coefficient which can be written as:

$${K}_{bs - px} = \frac{{S}_{px}}{{S}_{ems - det}} \cdot {\sum }_{i = 1}^{i + {N}_{layers}} ( {A}_{GFP - max} \cdot ( \frac{{T}_{exc}^{i - 1} ( Δ h )}{{E}_{ph} ( 470 nm )} ) \cdot {T}_{ems} ( {H}_{2 i} ) \cdot \frac{{Ω}_{a β i}}{4 π} \cdot {T}_{Fems 4 ems} ) \qquad ( EQ . 36 )$$

The relation between the quantified output (QO) of the light detector and the number of photons is given by:

$${N}_{pht} ( {T}_{int} ) = C \frac{( QO ( {T}_{int} ) - {QO}_{{DC\_e}^{-}} ( {T}_{int} ) ) / {2}^{bits}}{QE \cdot FF} , \qquad ( EQ . 37 )$$

where the quantified output due to dark current, QO_{DC}, can be measured by switching off the excitation light source.

Denoting the quantified GFP molecules emission by QI_{GFP}, the occupation of the fluorescent material can be written as:

$${n}_{GFP} = {QI}_{GFP} \cdot ( \frac{1}{{T}_{int} \cdot {P}_{fiber}} ) \cdot ( \frac{1}{{K}_{bs - px}} \cdot \frac{( 6 e 4 / {2}^{bits} )}{QE \cdot FF} ) \qquad ( EQ . 38 )$$

The separation of QI_{GFP} from QO_{int-sig}, can be done by logical division of the reaction chambers, in which in a first reaction chamber only the nutrition medium is placed (without the bacteria), hence generates only the contribution of I_{exc-Fem4exc}, I_{LB} and, I_{DC}. In a second reaction chamber both the nutrition medium and the bacteria are present hence generates, once interacting with the fluid sample, all the aforementioned contributions (i)-(iv).

The quantified emission intensity of the GFP molecules is thus obtained by subtraction:

QI_{GRP}=QO₂−QO₁ (EQ. 39)

where QO₁ and QO₁ are, respectively, the quantified output received from the first reaction chamber (without the bacteria) and the second reaction chamber (with the bacteria).

### Example 5

### Experimental

A prototype system was built according to the teaching of preferred embodiments of the invention described above. The prototype system included a sensing device with 12 reaction chambers and a plurality of micro-pumps were employed (see FIG. 3a), and a CMOS light detector (see Example 1). The sensors were E. coli-REC-A bacteria, referred to in this Example as biosensors.

The reaction chamber of the device were used as follows: (2 repetitions)×(2 biosensors E. coli−rec-A quantities: 1.5×10⁶, 1.5×10⁵ [bacteria/1 uL])×(3 Nalidixic Acid concentrations: 0, 5, 10 parts per million. The experiment duration was 5 hours.

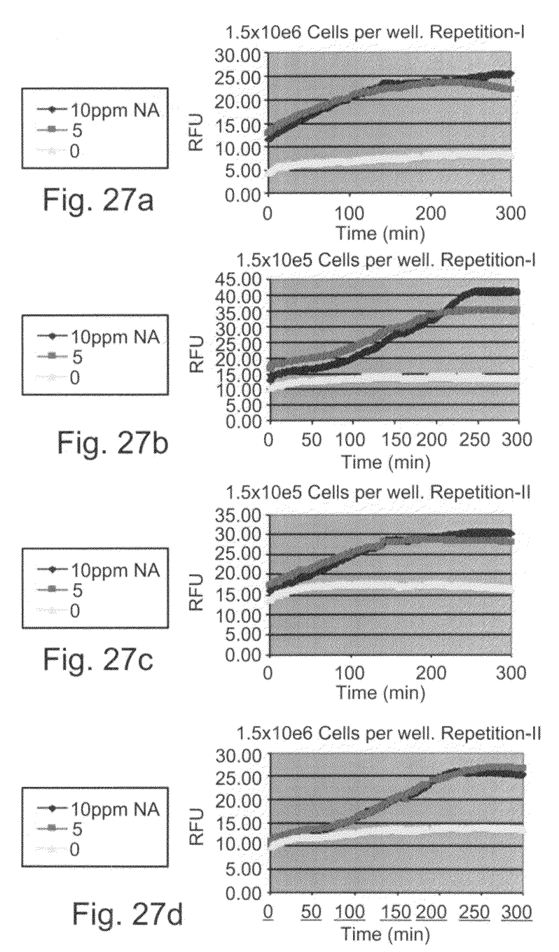

FIGS. 27a-d show the detected optical signal as a function of time. FIGS. 27a and 27c show the detected optical signal for a concentration of 1.5×10⁶ cells per reaction chamber (first and second repetitions, respectively), and FIGS. 27b and 27d show the detected optical signal for a concentration of 1.5×10⁵ cells per reaction chamber (first and second repetitions, respectively).

As shown in FIGS. 27a-d the strongest emission was registered for 0.5×10⁵ cells per reaction chamber. A significant biochemical reaction was acquired for 5 ppm and 10 ppm NA.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A portable system for detecting agents present in a sample, the system comprising:
a sensing device having a substrate formed with a plurality of reaction chambers and a plurality of channels interconnecting at least a portion of said plurality of reaction chambers, wherein at least a portion of said plurality of reaction chambers comprises a sensor, capable of generating a detectable signal when exposed to the agents;
a detector capable of receiving signals from said sensing device and providing an image of sensors generating said detectable signals; and
a communication unit, for connecting the portable system to a communication network.

2. A kit for detecting agents present in a sample, the kit comprising:
(a) a portable detection system, comprising:
a sensing device having a substrate formed with a plurality of reaction chambers and a plurality of channels interconnecting at least a portion of said plurality of reaction chambers, wherein at least a portion of said plurality of reaction chambers comprises a sensor, capable of generating a detectable signal when exposed to the agents;
a detector capable of receiving signals from said sensing device and providing an image of sensors generating said optical signals; and
a communication unit, for connecting the portable system to a communication network; and
(b) a sampling device being connectable to said detection system and capable of sampling fluids from the environment and delivering said fluids to said sensing device.

3. The kit of claim 2 wherein said sampling device comprises a plurality of containers and treating elements for treating fluids in said plurality of containers prior to said transfer of said fluids to said sensing device.

4. The kit of claim 2, further comprising a medical injection device, for injecting at least one medicament to a mammal.

5. The kit of claim 4, wherein said injection device comprises:
an injection needle;
a plurality of syringes, each having a plunger slidably disposed therein;
a driving mechanism having a plurality of drive members, each drive member of said plurality of drive members being operable to engage a plunger of a respective syringe of said plurality of syringes; and
a redirector for redirecting medicaments, released by at least one syringe of said plurality of syringes, into said injection needle.

6. The kit of claim 2, further comprising a storage unit for storing said sensing device, said sampling device and/or said fluids, and maintaining a predetermined temperature thereof.

7. The kit of claim 6, wherein said storage unit comprises:
at least one cavity having a plurality of temperature control units and configured to receive a plurality of objects in a manner such that a plurality of thermal communications are established between said plurality of temperature control units and said plurality of objects, each of said plurality of objects being selected from the group consisting of said sensing device, said sampling device and said fluids; and
a controller, configured to receive temperature information from said plurality of temperature control units and to independently activate each temperature control unit to maintain a predetermined temperature of a respective object.

8. A method of distributing information of a presence of agents in the environment, the method comprising:
sampling the environment thereby providing a sample;
delivering said sample through a plurality of channels to thereby contact said sample with at least one sensor, capable of generating detectable signals when exposed to the agents;
upon a detection of said detectable signals, processing said detectable signals so as to determine presence or level of an agent in said sample; and
transmitting signals indicative of said presence or level of said agent over a communication network.

9. The method of claim 8, further comprising determining a location at which said sampling is performed and transmitting said location over said communication network.

10. The method of claim 8, further comprising continuously monitoring environmental conditions, wherein said sampling is performed when said environmental conditions meet a predetermined set of criteria.

11. The method of claim 8, further comprising transmitting an identification code over said communication network.

12. The method of claim 8, further comprising detection presence of agents in the environment by a process selected from the group consisting of optical detection, gas chromatography, mass spectroscopy, time of flight analysis and any combination thereof.

13. The method of claim 8, further comprising monitoring atmospheric condition and transmitting said atmospheric condition over said communication network.

14. The method of claim 8, further comprising capturing an image of at least a portion of the environment and capturing and transmitting said image over said communication network.

15. The method of claim 8, wherein said sample is a liquid sample.

16. The method of claim 8, wherein said sample is a gas sample.

17. The method of claim 8, wherein said sample is a solid sample.

18. The method of claim 8, further comprising binding components of said solid sample to a liquid phase.

19. A medical injection device, comprising:
an injection needle;
a plurality of syringes, each having a plunger slidably disposed therein;
a driving mechanism having a plurality of drive members, each drive member of said plurality of drive members being operable to engage a plunger of a respective syringe of said plurality of syringes;
a redirector for redirecting medicaments, released by at least one syringe of said plurality of syringes, into said injection needle.

20. The device of claim 19, further comprising a communication unit, operable to communicate a communication network, wherein said driving mechanism is designed and constructed to receive activation signals from said communication unit, to thereby activate said at least one syringe.

21. A distributed detection system for detection of the presence of harmful agents in an environment, comprising a central monitoring unit and a plurality of portable agent detection systems being mounted on mobile vectors for release into said environment, each portable agent detection system of said plurality of portable agent detection systems comprising:
a sensing device having a substrate formed with a plurality of reaction chambers and a plurality of channels interconnecting at least a portion of said plurality of reaction chambers, wherein at least a portion of said plurality of reaction chambers comprises a sensor, capable of generating a detectable signal when exposed to the agents;
a detector capable of receiving signals from said sensing device and providing an image of sensors generating said optical signals; and
a communication unit, for connecting the portable system to a communication network.

22. The system of claim 1 wherein said communication unit is designed to transmit signals representing presence, level or absence of the agents.

23. The system of claim 1 wherein said portable detection system further comprises a positioning unit, for determining a location of the system, wherein said communication unit is designed to transmit said location over said communication network.

24. The system of claim 1 wherein said portable detection system further comprises an activation unit for activating or selecting an operational mode of the system.

25. The system of claim 1 being identifiable by an identification code, and further wherein said communication unit is operable to transmit said identification code over said communication network.

26. The system of claim 1 wherein said portable detection system further comprises detection hardware.

27. The system of claim 1 wherein said portable detection system further comprises vital signs measuring unit for measuring vital signs of a mammal carrying the system.

28. The system of claim 1 wherein said portable detection system further comprises an atmospheric condition measuring unit for measuring at least one atmospheric condition.

29. The system claim 1 wherein said portable detection system further comprises an image capturing unit.

30. The system of claim 1 wherein said portable detection system further comprises an input-output audio unit.

31. The system of claim 1 wherein said communication unit is supplemented with at least one communication protocol, tangibly embodied in a readable memory, said at least one communication protocol being configured to allow a takeover of said communication network.

32. The system of claim 1 wherein said portable detection system further comprises a nucleic acid amplification unit.

33. The system of claim 1 wherein said portable detection system further comprises a shape detector.

34. The system of claim 1 wherein said portable detection system further comprises a motion detector.

35. The system of claim 1 wherein said portable detection system further comprises a data processor, supplemented by an algorithm for receiving image information from said detector and determining presence or level of the agents.

36. The system of claim 1 wherein said portable detection system further comprises a control unit for sending control signals to said sensing device.

37. The system of claim 1 wherein said portable detection system further comprises a temperature control unit for controlling a temperature of said sensing device and/or said detector.

38. The system of claim 1 wherein said sensing device is removable.

39. The system of claim 1 wherein said sensing device is disposable.

40. The system of claim 1 wherein said sensor is a biological sensor.

41. The system of claim 40, wherein said biological sensors is capable of producing a bioluminescent material.

42. The system of claim 40, wherein said biological sensors is capable of producing a phosphorescent material.

43. The system of claim 40, wherein said biological sensor is capable producing a fluorescent material.

44. The system of claim 1 wherein said portable detection system further comprises a transport mechanism for actuating transport of a sample fluid in said plurality of fluid channels, thereby to fill said plurality of reaction chambers with said sample fluid.

45. The system of claim 1 wherein said detector is capable of providing said image substantially in real time.

46. The system of claim 45, wherein a portion of said plurality of reaction chambers comprises a material capable of generating a detectable reference signal at all times.

47. The system of claim 1 wherein said sample is a liquid sample.

48. The system of claim 1 wherein said sample is a gas sample.

49. The system of claim 1 wherein said sample is a solid sample.

50. The system of claim 40, wherein said biological sensors comprises a population of cells, said population of cells including a reporter expression construct being expressible in a cell of said population when exposed to the agent.

51. A storage unit, comprising:
at least one cavity having a plurality of temperature control units and configured to receive a plurality of objects in a manner such that a plurality of thermal communications are established between said plurality of temperature control units and said plurality of objects; and
a controller, configured to receive temperature information from said plurality of temperature control units and to independently activate each temperature control unit to maintain a predetermined temperature of a respective object.
